(19) United States
(12) Patent Application Publication
(10) Pub. No.: US 2026/0041747 A1
(43) Pub. Date: Feb. 12, 2026

(54) T CELL EPITOPES ASSOCIATED WITH TYPE 1 DIABETES

(71) Applicant: Repertoire Immune Medicines, Inc., Cambridge, MA (US)

(72) Inventors: William Edward RUFF, JR., Cambridge, MA (US); John PAJEROWSKI, Cambridge, MA (US)

(73) Assignee: Repertoire Immune Medicines, Inc., Cambridge, MA (US)

(21) Appl. No.: 19/099,435

(22) PCT Filed: Jul. 28, 2023
(86) PCT No.: PCT/US2023/071202

§ 371 Date: Jan. 28, 2025

Related U.S. Application Data
(60) Provisional application No. 63/393,616, filed on Jul. 29, 2022.

(51) Int. Cl. A61K 39/00; A61K 31/07; A61K 31/197; A61K 31/427; A61K 31/593; A61K 31/7076; A61K 38/08; A61K 39/385; A61K 47/69; A61P 37/04; C07K 14/55

(52) U.S. Cl. CPC A61K 39/0008; A61K 31/07; A61K 31/197; A61K 31/427; A61K 31/593; A61K 31/7076; A61K 38/08; A61K 39/385; A61K 47/6911; A61P 37/04; C07K 14/55; A61K 2039/53; A61K 2039/55555; A61K 2039/605

(57) ABSTRACT

Provided herein are T cell epitopes associated with Type 1 diabetes. Also provided are antigen-presenting cells presenting such epitopes. T cells reactive to such epitopes, and related compositions and therapies.

### CROSS-REFERENCE TO RELATED APPLICATIONS

This application claims priority to U.S. Provisional Application No. 63/393,616 filed Jul. 29, 2022, the entire contents of which is hereby incorporated by reference.

### REFERENCE TO SEQUENCE LISTING SUBMITTED ELECTRONICALLY

The content of the electronically submitted sequence listing (Name: 4985_033PC01_SequenceListing_ST26.xml, created on Jul. 18, 2023 and having a size of 122,947 bytes) is herein incorporated by reference in its entirety.

### FIELD OF THE DISCLOSURE

The disclosure relates to T cell epitopes associated with Type 1 Diabetes (T1D). The disclosure also relates to antigen-presenting cells (APCs) presenting such epitopes, T cells reactive to such epitopes, and related compositions and therapies.

### BACKGROUND

Type 1 Diabetes (T1D) is a prevalent chronic disease with about 1.6 million adult and pediatric patients in the United States, and about 64,000 new cases diagnosed per year. T1D is an autoimmune disease characterized by T-cell-mediated destruction of pancreatic β cells, resulting in a deficiency of insulin synthesis and secretion. To manage the disease, patients are dependent on insulin substitution therapy for the rest of their lives. To date, there is no cure for T1D. The standard of care treatment for T1D includes administration of insulin, metformin, Pramlintide (SYMLIN®), blood pressure drugs, cholesterol medications, and aspirin, as well as exercise and diet. The first and only disease modifying drug for T1D was FDA approved in 2022 for the delay of T1D, TZIELD™, no other disease modifying therapy is approved for T1D.

Despite advances in treatment modalities for T1D patients, the majority of T1D patients have a lower life expectancy and are at a chronic risk for diabetic coma or ketoacidosis. Long term T1D damages the heart and blood vessels, mainly by promoting high blood pressure and atherosclerosis that can lead to other diabetes-related complications, such as heart disease, chronic kidney disease, nerve damage, and other problems with feet, oral health, vision, hearing, and mental health. Accordingly, there remains a need for new treatments for these patients.

### SUMMARY OF THE DISCLOSURE

The present disclosure is based, in part, upon the identification of immunoprevalent T cell epitopes associated with Type 1 Diabetes (T1D). It has been discovered that CD8⁺ T cells that bind these epitopes are present at high frequencies across T1D patients and at low frequencies across healthy donors. Furthermore, some of these epitopes have also been found to induce a robust response from CD8⁺ T cells of T1D patients. Accordingly, T1D-associated peptides comprising one or more of these T cell epitopes can be used for inducing immune tolerance to cells (e.g., islet β cells) presenting these epitopes.

Accordingly, in one aspect, the present disclosure provides a composition (e.g., pharmaceutical composition) comprising a T1D-associated peptide (e.g., an isolated T1D-associated peptide) comprising a T cell epitope amino acid sequence set forth in Table 1, Table 2, or Table 3, or a nucleic acid encoding the T1D-associated peptide. Depending on the use, the peptide can comprise 8-100, 8-50, 8-30, 8-25, 8-20, 8-15, 15-100, 15-50, 15-30, 15-25, or 15-20 contiguous amino acids of the corresponding antigen set forth in Table 1, Table 2 or Table 3. Specifically contemplated are T1D-associated peptides comprising 8-100, 8-50, 8-30, 8-25, 8-20, 8-15, 15-100, 15-50, 15-30, 15-25, or 15-20 contiguous amino acids of a corresponding antigen set forth in Table 2. Also contemplated are T1D-associated peptides comprising various lengths, for example, 8-100, 8-50, 8-30, 8-25, 8-20, 8-15, 15-100, 15-50, 15-30, 15-25, or 15-20 contiguous amino acids, of a corresponding antigen set forth in Table 1.

The pharmaceutical composition can further comprise a vehicle for targeting the T1D-associated peptide or the nucleic acid to a target cell. These peptides and/or nucleic acids encoding these peptides can be used in a variety of therapeutic modalities.

In a first therapeutic modality, the vehicle comprises a delivery agent for delivering the T1D-associated peptide or the nucleic acid into the target cell. It is contemplated that the peptide, either delivered by the delivery agent or produced from a nucleic acid delivered by the delivery agent, is processed within the target cell and presented by a cognate MHC on the surface of the target cell. The target cell, which has a tolerogenic character (naturally possessed, induced by an immunomodulator in the pharmaceutical composition, or both), can then induce immune tolerance to cells (e.g., islet β cells) that present these T cell epitopes.

With respect to the T1D-associated peptide, the peptide can comprise 8-100, 8-50, 8-30, 8-25, 8-20, 8-15, 15-100, 15-50, 15-30, 15-25, or 15-20 contiguous amino acids of the corresponding antigen set forth in Table 2, Table 1, or Table 3. Additionally or alternatively, the T1D-associated peptide can comprise at least 70%, at least 80%, at least 90%, or at least 95% of the entire length of the corresponding antigen. Where the composition comprises the T1D associated peptide, the delivery agent can comprise a nanoparticle. For example, the nanoparticle can comprise a liposome. Where the composition comprises the nucleic acid encoding the T1D associated peptide, the nucleic acid can be a messenger RNA (mRNA). Additionally, the delivery agent can comprise a lipid nanoparticle.

With respect to the target cell, the target cell targeted by the delivery agent can be a dendritic cell, a macrophage, a B cell, or a non-professional antigen presenting cell (APC). For example, the target cell can be a tolerogenic cell. For example, the tolerogenic cell can be a cell in the liver, optionally selected from a liver sinusoidal endothelial cell, a MARCO+ Kupffer cell, and a monocyte-derived macrophage. Alternatively, the tolerogenic cell can be a cell in the spleen, optionally selected from a marginal zone macrophage, a metallophilic macrophage, and a marginal zone dendritic cell.

With respect to the delivery agent, the delivery agent can have negative zeta potential. The delivery agent can further comprise additional components, for example a ligand that binds the target cell.

The pharmaceutical composition can further comprise an immunomodulator that can be delivered to the target cell or the microenvironment of the target cell. For example, the immunomodulator can comprise an immunomodulatory cytokine or a nucleic acid encoding the immunomodulatory cytokine. The immunomodulatory cytokine can comprise, for example, IL-2, IL-10, TGF-β, IL-37, IL-27, IL-35, IL-31 or Vasoactive Intestinal Peptide (VIP), or variants thereof. Where the immunomodulatory cytokine is a mutant IL-2, it may preferentially activate the IL-2Rαβγ receptor complex relative to the IL-2Rβγ receptor complex. Additionally or alternatively, the immunomodulator can comprise a nucleic acid encoding an intracellular or transmembrane immunomodulatory protein. The intracellular or transmembrane immunomodulatory protein can comprise, for example, PD-L1, PD-L2, ICOS ligand, ILT3, ILT4, BTLA, Fas, CD39, or indoleamine 2,3-dioxygenase 1 (IDO1). Further, additionally or alternatively, the immunomodulator can comprise an immunomodulatory compound. The immunomodulatory compound can comprise, for example, vitamin A, vitamin D, adenosine, kynurenine, or 2-(1′ H-indole-3′-carbonyl)-thiazole-4-carboxylic acid methyl ester (ITE). For targeted delivery to the target cell, the immunomodulator can be in or on the delivery agent.

In a second therapeutic modality, the composition comprises a peptide-MHC (pMHC) complex comprising the T1D-associated peptide and a cognate class I MHC. Given that, under certain circumstances, pMHC complexes can have low binding affinities to cognate T cell receptors (TCRs), a presentation agent can be used to multimerize or cluster pMHC complexes to increase their binding avidity to a TCR. Therefore, the vehicle in the second therapeutic modality can comprise a presentation agent that binds a plurality of the pMHC complexes disclosed herein, where the presentation agent allows efficient targeting of the pMHC complex to a T cell that expresses a cognate TCR, e.g., a cytotoxic T cell that mediates T1D progression. The second therapeutic modality can further comprise an immunomodulator, which may suppress survival, proliferation, activation, and/or memory formation the T cell.

With respect to the T1D-associated peptide, the peptide can consist of an amino acid sequence set forth in Table 1, Table 2, or Table 3. With respect to the class I MHC, the class I MHC can be the HLA-A*02:01 allele. The T1D-associated peptide may bind a cognate class I MHC via α1 and α2 domains of the MHC. Also contemplated are soluble class I MHCs, that can comprise an α1 domain and an α2 domain. Additionally, the soluble class I MHC can comprise an α3 domain and a β2-microglobulin (β2m) subunit. It is further contemplated that the T1D-associated peptide can be N-terminally or C-terminally fused to the β2m subunit. For presentation of the pMHC to the target cell, the peptide-MHC complex can be presented by a variety of entities. For example, the pMHC can be located on an outer surface of the presentation agent. Also contemplated are compositions that comprise an antigen presenting cell (APC) for presenting the MHC. APCs useful in the composition comprise artificial antigen presenting cell (aAPC), for example nanoparticles. Also contemplated is a presentation agent that comprises a multimerization domain linked to the peptide-MHC complex. The pharmaceutical composition can further comprise an immunomodulator that can be delivered to the target cell or the microenvironment of the target cell. For example, the immunomodulator can comprise an immunomodulatory cytokine. The immunomodulatory cytokine can comprise, for example, IL-2, IL-10, TGF-β, IL-37, IL-27, IL-35, IL-31, and Vasoactive Intestinal Peptide (VIP), or variants thereof. Additionally or alternatively, the immunomodulator can comprise an agonist of an immunomodulatory receptor of a T cell. For example, the immunomodulator can comprise an agonist of PD-1, 4-1BB, CTLA-4, BTLA, LAG-3, TIM-3, TIGIT, CD2, or CD3. To aid with delivery to the target cell, it is further contemplated that the immunomodulator can be conjugated to the peptide-MHC complex or the presentation agent.

These therapeutic modalities (e.g., the first or the second therapeutic modality) can be comprised in a pharmaceutical composition. The pharmaceutical composition can further comprise a pharmaceutically acceptable carrier or excipient.

Methods of treatment for T1D are also contemplated. In this aspect, the present disclosure provides a method of treating T1D in a subject, the method comprising administering to the subject a therapeutically effective amount of the pharmaceutical composition. With respect to the patient, it is contemplated that the patient expresses a class I MHC HLA-A*02:01 allele. Tolerance of the subject to T1D-associated peptides can be induced by the methods of treatment, for example, by generating tolerogenic APCs that present the peptides in vivo or by suppressing survival, proliferation, activation, and/or memory formation cognate T cells (e.g., cytotoxic T cells).

In some aspects, the disclosure provides means for inducing a tolerogenic immune response in a subject in need thereof, in combination with a pharmaceutically acceptable carrier. In some aspects, the disclosure is directed to a method of treating or preventing type I diabetes in a subject in need thereof comprising administering to the subject an effective amount of a means for inducing a tolerogenic immune response in combination with a pharmaceutically acceptable carrier.

### BRIEF DESCRIPTION OF THE FIGURES

FIG. 1 depicts a dose-response for the islet-derived rTCR described in Table 5 when pulsed with a range of cognate peptide from 0.001 to 100 μM. Based on this response curve an EC50 (half maximal effective concentration) of 2.2 μM could be determined.

FIG. 2 depicts 50 μM peptide pulsed pMHC-rTCR responses relative to control DMSO pulsed APCs. Shown to the left of the dashed line are rTCRs with confirmed or validated ability to produce activation signals upon ligation to cognate pMHC above background, which are listed in Table 6.

### DETAILED DESCRIPTION

The present disclosure is based, in part, upon the identification of immunoprevalent T cell epitopes associated with Type 1 Diabetes (T1D) and use of such T cell epitopes or a nucleotide sequence encoding such T cell epitopes to induce immune tolerance to cells, e.g., pancreatic cells. It has been discovered that CD8⁺ T cells that bind these epitopes are present at high frequencies across T1D patients and at low frequencies across healthy donors. Certain of these epitopes have also been found to induce a robust response from CD8⁺ T cells of T1D patients. Accordingly, T1D-associated peptides comprising these T cell epitopes can be used for inducing immune tolerance to cells (e.g., pancreatic cells, e.g., islet β cells) presenting these epitopes.

### I. Definitions

To facilitate an understanding of the present disclosure, a number of terms and phrases are defined below.

As used herein, the terms “a” and “an” mean “one or more” and include the plural unless the context is inappropriate.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

As used herein, the expression “and/or” in connection with two or more recited objects includes individually each of the recited objects and the various combinations of two or more of the recited objects, unless otherwise understood from the context and use.

As used herein, the terms “antigen-presenting cell” or “APC” refer to a cell or particle that elicits a cellular immune response by displaying a T cell epitope presented by a major histocompatibility complex (MHC) on an outer surface of the cell or particle, for recognition by an immune cell such as a T cell. APCs include, e.g., professional APCs, such as dendritic cells, macrophages, Langerhans cells, and B cells, that express both class I and class II MHCs, and non-professional APCs (e.g., nucleated cells) that generally express only class I MHCs. APCs also include artificial APCs (aAPCs), e.g., cells (e.g., drosophila cells) engineered to express an MHC that presents a T cell epitope. In addition, artificial APCs include particles (e.g., beads, nanoparticles, or liposomes) that present a peptide-MHC on the surface, configured such that the aAPC can prime or stimulate T cells in vitro, ex vivo, or in vivo. Exemplary aAPCs are known in the art and are described in Section V herein.

As used herein, the term “delivery agent” refers to an agent capable of delivering a cargo (e.g., peptide, protein, nucleic acid, or other compounds) to a target cell, tissue, or organ. In one example, a delivery agent may deliver a cargo into a target cell (e.g., cytosol or endosome of the target cell). In another example, a delivery agent may deliver a cargo to the vicinity of a target cell in a target tissue, such that the cargo can bind to an outer surface of the target cell or be internalized into the target cell. A delivery agent may, e.g., bind to the cargo or encapsulate the cargo. Delivery agents include, for example, particles such as nanoparticles (e.g., lipid nanoparticles), exosomes, lipid-like particles, vectors, microvesicles, or liposomes (e.g., nanoliposomes).

As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route.

As used herein, percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Similarly, percent “identity” between a nucleic acid sequence and a reference sequence is defined as the percentage of nucleotides in the nucleic acid sequence that are identical to the nucleotides in the reference sequence, after aligning the sequences and introducing gaps, if necessary, to achieve the maximum percent sequence identity. Alignment for purposes of determining percent sequence identity (e.g., amino acid sequence identity or nucleic acid sequence identity) can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

As used herein, the term “isolated” when used in reference to a biological substance means that the substance is not in its native state or is removed from at least a portion of other molecules associated or occurring with the substance in its native environment (e.g., within a cell or tissue) or in another environment (e.g., in a cell extract, extraction buffer, etc.). An isolated biological substance, e.g., a protein, peptide, nucleic acid, or cell, can be essentially free of other biological molecules. For example, in certain embodiments, an isolated protein, peptide, nucleic acid, or cell can be at least 40%, 50%, 60%, 70%, 80%, 90%, 95%, 96%, 97%, 98%, 99% or 100% free of other molecules present in a natural or non-natural environment. For example, in certain embodiments, an isolated peptide is not present in a native composition of the peptide, e.g., in a cell comprising the peptide in its cytoplasm, or an isolated antigen-presenting cell (“APC”) is not present in a native environment of the APC, e.g., in a biological sample (e.g., tissue or body fluid sample) comprising the APC.

The term “major histocompatibility complex” or “MHC” refers to a genomic locus containing a group of genes that encode the polymorphic cell-membrane-bound glycoproteins known as MHC class I and class II molecules, which regulate the immune response by presenting peptides of fragmented proteins to CD8⁺ (e.g., cytotoxic) and CD4⁺ (e.g., helper) T lymphocytes, respectively. In humans, this group of genes is also called “human leukocyte antigen” or “HLA.” Human MHC class I genes encode, for example, HLA-A, HLA-B, and HLA-C proteins. Class I HLA protein is a heterodimer composed of a heavy α-chain and a smaller β-chain. The α-chain is encoded by a variant HLA gene, and the β chain is an invariant β2 microglobulin (β2m) polypeptide encoded by a separate region of the human genome. Human MHC class II genes encode, for example, HLA-DM, HLA-DO, HLA-DP, HLA-DQ, and HLA-DR proteins. Class II HLA protein is a heterodimer composed of α and β chains both encoded by variant HLA genes. A HLA can include multiple serotypes (e.g., HLA-A*02, HLA-DPA1*02) which each may include multiple alleles (e.g., HLA-A*02:01, HLA-DPA1*02:02). Nucleotide sequences and a gene map of human MHC are publicly available (e.g., The MHC sequencing consortium, Nature 401:921-923, 1999). The terms “major histocompatibility complex” and “MHC” also refer to the polymorphic glycoproteins encoded by the MHC class I or class II genes, where appropriate in the context, and proteins comprising variants thereof that bind T cell epitopes (e.g., class I or class II epitopes). Generally, a class I MHC binds a T cell epitope in a groove formed by its α1 domain and α2 domain and a class II MHC binds a T cell epitope in a groove formed by its α1 domain and β1 domain. The term “soluble MHC” refers to an extracellular fragment of a MHC comprising corresponding α1 and α2 domains that bind a class I T cell epitope or corresponding α1 and β1 domains that bind a class II T cell epitope, where the α1 and α2 domains or the α1 and β1 domains are derived from a naturally occurring MHC or a variant thereof.

As used herein, the term “pharmaceutical composition” refers to the combination of an active agent with a carrier (e.g., a pharmaceutically acceptable carrier), inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Adejare, Remington, The Science and Practice of Pharmacy (23rd Ed. 2020).

As used herein, the term “presentation agent” refers to an agent (e.g., a cell or particle) that can bind a plurality of peptide-MHC complexes (e.g., a plurality of the same peptide-MHC complexes) for recognition by a TCR, e.g., a TCR expressed by a T cell. Presentation agents include but are not limited to APCs (e.g., dendritic cells or macrophages), artificial APCs (e.g., engineered cells, nanoparticles, or liposomes), and multimerization domains linked to peptide-MHC complexes.

As used herein, the terms “prime” and “priming,” in the context of antigen presenting cells and T cells, refers to naïve T cell clonal expansion, activation, and/or generation of memory T cells as a result of TCR engagement by a T cell epitope presented by a MHC on an outer surface of an antigen presenting cell.

As used herein, the terms “subject” and “patient” are used interchangeably and refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “target cell” refers to a cell that can be targeted by a delivery agent. For example, a target cell may have a cell surface receptor (e.g., a TCR) that can be bound by the delivery agent, e.g., via a ligand present on the delivery agent that binds the cell surface receptor. In another example, a target cell may be located in a specific tissue or organ (e.g., liver, lymph nodes, or spleen) that the delivery agent is enriched in or delivered to. A target cell may comprise, e.g., an immune cell (e.g., dendritic cell or macrophage).

As used herein, a cell composition is “substantially free” of a specified type of cells if this type of cells does not exceed 0.1%, 0.01%, or 0.001% of the total amount of cells in the composition. The percentage of each cell type in a cell composition can be determined using a method known in the art. For example, immune cells are often characterized by cell type-specific markers (e.g., cell surface markers) and the amount of each cell type can be determined by flow cytometry.

As used herein, the term “T cell epitope” refers to a peptide of an antigen protein presentable by a class I or class II MHC. T cell epitopes presentable by class I MHC proteins (e.g., HLA-A, HLA-B) are typically 8-11 amino acids in length. The peptide is located in the peptide-binding groove of the central region of the α1/α2 domains of the class I MHC. T cell epitopes presentable by class II MHC proteins are typically 13-25 amino acids (e.g., 15-24 amino acids) in length. The peptide is located in the peptide-binding groove formed by the α1/β1 domains of the class II MHC. A T cell epitope is “immunogenic,” relative to a reference T cell epitope, if the T cell epitope stimulates a stronger and/or longer-lasting T cell response than the reference T cell epitope in a subject, for example, as measured by the amount of total T cells and/or memory T cells, respectively, reactive to the T cell epitope. The strength and duration of the T cell response (e.g., proliferation or activation) can be specific to the subject's MHC serotypes and TCR repertoire. Immunogenic T cell epitopes can also be identified in a population of organisms (e.g., humans) with respect to its average strength and duration of T cell response to the T cell epitopes. A T cell epitope is “immunoprevalent” when the number of T cells binding to the epitope is increased in a population or subpopulation, relative to a reference T cell epitope interaction or a group of T cell epitope interactions. Immunoprevalence can be specific to the T cell population or subpopulation of interest. For example, a T cell epitope immunoprevalent at a site of tissue or lesion may not be immunoprevalent in peripheral blood. It is understood that immunogenicity and immunoprevalence are not necessarily equivalent. For example, an immunogenic T cell epitope may correspond to a rare T cell population and therefore is not immunoprevalent. Alternatively, an immunoprevalent T cell epitope may only stimulate a weak T cell activation.

As used herein, the terms “T cell receptor” and “TCR” refer to a surface protein (e.g., a heterodimeric protein) of a T cell that allows the T cell to recognize an antigen and/or an epitope thereof, typically presented by a major histocompatibility complex (MHC), or a fragment of such surface protein comprising at least its variable domains. Typically, TCRs are heterodimers comprising two different protein chains. In many T cells, the TCR comprises an alpha (a) chain and a beta (B) chain. Each chain, in its native form, typically comprises two extracellular domains, a variable (V) domain and a constant (C) domain, the latter of which is membrane-proximal. The variable domain of α-chain (Va) and the variable domain of β-chain (VB) each comprise three hypervariable regions that are also referred to as the complementarity determining regions (CDRs) such as CDR1, CDR2, and CDR3. The CDRs, in particular CDR3, are primarily responsible for contacting epitopes and thus define the specificity of the TCR, although, under certain circumstances, the CDR1 of the α-chain can interact with the N-terminal part of the antigen, and CDR1 of the β-chain interacts with the C-terminal part of the antigen.

As used herein, the term “treating” includes any effect, e.g., lessening, reducing, modulating, ameliorating or eliminating, that results in the improvement of the condition, disease, disorder, and the like, or ameliorating a symptom thereof. It is understood that T1D can be diagnosed by assessing genetic, immunologic, and/or metabolic markers prior to symptomatic onset. As used herein, the term “treating” also includes prevention of symptomatic onset after observation of the predictive markers.

As used herein, the term “vehicle” refers to an agent (e.g., delivery agent or presentation agent) that can bind or contain a biomolecule (e.g., a peptide, protein, or nucleic acid). For example, a vehicle can deliver a biomolecule to a target cell, tissue, or organ. In another example, a vehicle can bind one or more peptide-MHC complexes for recognition by a TCR. It is contemplated that in certain embodiments, the vehicle that binds or contains the biomolecule is not naturally occurring.

As a general matter, compositions specifying a percentage are by weight unless otherwise specified. Further, if a variable is not accompanied by a definition, then the previous definition of the variable controls.

### II. T Cell Epitopes and Cognate MHCS

T cell-mediated immune responses are generally employed to kill undesired cells, such as cancer cells and pathogen-infected cells. T cells reactive to autoantigens are generally removed by negative selection in the thymus, an organ that shrinks in adulthood. However, failure to remove autoreactive T cells, among other mechanisms, can lead to T cell attack of healthy cells or tissues, resulting in autoimmune diseases (e.g., T1D).

Unlike humoral immune responses, T cell immunity relies on T cell receptor (TCR) recognition of antigenic peptides presented by MHCs. While only particular epitopes of surface proteins allow targeting by neutralizing antibodies, many peptides can serve as targets of T cells. Peptides from antigens delivered endogenously to APCs (e.g., expressed in the APCs) are processed primarily for association with class I MHC. Peptides from antigens delivered exogenously to APCs (e.g., internalized by endosomes) are processed primarily for association with class II MHC. Thus, T cell immunity can be elicited towards antigen proteins having various physiological functions and localizations.

T cell epitopes of a given antigen can be predicted by in silico methods, for example, as described in Schaap-Johansen et al., (2021) Front. Immunol. 12:712488. Experimental methods have also been developed to verify presentability of a T cell epitope with respect to a given MHC (see, e.g., WO2019197671). However, these methods generally focus on MHC presentation, whereas the immunogenicity and immunoprevalence of a T cell epitope still needs to be determined empirically. Various methods of identifying immunogenic and immunoprevalent T cell epitopes have been described in the art. For example, a method has been developed to identify immunoprevalent T cell epitopes from a library of peptide-MHC-TCR fusion (MCR) proteins using human-derived T cell receptors (see Kisielow et al., Nat. Immunol. (2019) 20:652-62; WO2016097334). Each MCR protein includes a peptide, an extracellular domain of a MHC allele, and transmembrane and intracellular domains of a TCR. Reporter T cells are engineered to constitutively express the library of MCR proteins and to express a reporter gene (e.g., GFP) under the control of a TCR-responsive transcription factor (e.g., NFAT). When contacting the reporter T cells and T cells from a patient sample, a reporter T cell expresses the reporter gene if the peptide and MHC of its MCR protein is recognized by a TCR of a patient T cell. By assessing a large number of patient samples, this system can be utilized for identification of immunoprevalent T cell epitopes in the library of MCRs that are associated with the disease. For example, this screening platform, in which the disclosure is based has identified class I-restricted anti-cancer T cells in HPV positive oropharyngeal cancers (see McInnis et al., (2023) J Immunother Cancer 11:3 (PMID: 36990508)). Therefore, in some aspects the same screening technique can be used to identify disease-relevant T cells, such as T1D-relevant T cells.

The ability of a T cell epitope to bind T cells (e.g., T cells of T1D patients) can be assessed using a peptide-MHC multimer (e.g., tetramer) including the T cell epitope. Such multimers, incorporating the T cell epitope, a soluble fragment of MHC, and optionally a marker (e.g., fluorophore or barcode), can be produced by various methods in vitro, for example, as described in Jordan et al., J. Immunol. (2008) 180(1): 188-97, WO2021202727; and WO2021262872. Briefly, a T cell sample can be incubated with a library of peptide-MHC (“pMHC”) complex. The T cells that bind the pMHC can then be isolated and the peptides identified. The method can be conducted in a multiplex manner. For example, a plurality of barcoded pMHC multimers can be contacted with T cells in parallel, followed by replicating each TCR-encoding nucleic acid in a hydrogel linked with a barcode (see, e.g., WO2020142724 and WO2021138588).

One of the challenges to developing T cell-directed T1D therapies is identification of strongly immunogenic T cell epitopes that contribute to T1D pathogenesis. It has been discovered that CD8⁺ T cells that bind the T cell epitopes set forth in Table 1, Table 2, and Table 3 are present at high frequencies across T1D patients and at low frequencies across healthy donors (see Example 2). Some of these epitopes have also been found to induce a robust response from CD8⁺ T cells of T1D patients (see Example 3). Accordingly, the present disclosure provides the T cell epitopes set forth in Table 1, Table 2, and Table 3, nucleotides encoding the T cell epitopes, and compositions comprising these epitopes or the nucleotides, that may be useful in T cell-directed T1D therapies. These T cell epitopes are presentable by HLA-A*02:01 and are immunoprevalent across patients who express HLA-A*02:01.

TABLE 1
Exemplary T cell epitopes Associated with T1D

| | Epitope | SEQ |
|---|---|---|
| Antigen | sequence | ID NO |
| IFIH1 | KLLEIMTRI | 1 |
| GCG | FVAGLFVML | 2 |
| EGR4 | LMSGILGLA | 3 |
| BMP5 | QALDVGWLV | 4 |
| EGR4 | YAPWELLSV | 5 |
| C1ORF127 | ALAWAVWLA | 6 |
| EGR4 | FLSWALNSC | 7 |
| PDX1 | GQWAGGAYA | 8 |
| SH2B3 | NLYTFVLKV | 9 |
| RASGRP1 | TLMAVIGGL | 10 |
| CYP27B1 | TLSWALYEL | 11 |
| IFIH1 | VLVNKVLLV | 12 |
| CYP27B1 | TLVTLCHYA | 13 |
| FUT2 | KMNGRPAFI | 14 |
| REG1B | AQTNSFFML | 15 |
| CPA1 | GLLVLSVLL | 16 |
| BMP5 | HMNATNHAI | 17 |
| PLA2G1B | KMIKCVIPG | 18 |
| CLPS | LLLVALSVA | 19 |
| C1ORF127 | RLAAPLAVL | 20 |
| CPA1 | SIIKAIYQA | 21 |
| C1ORF127 | SLEAACPPV | 22 |
| GJD2 | TILERLLEA | 23 |
| C1ORF127 | TLWIPRSHV | 24 |
| GJD2 | TMIGRILLT | 25 |
| C1ORF127 | YMTLWIPRS | 26 |
| CPA1 | TLDIFLEIV | 27 |
| C1ORF127 | YVDMNATTV | 28 |
| CVB4E2 | ALSTCYIMCMV | 29 |
| DLK1 | ALRPYLPAL | 30 |
| CYP27B1 | AMPHWLRHL | 31 |
| RASGRP1 | ASFPFSFCV | 32 |
| IFIH1 | CMEEELLTI | 33 |
| FUT2 | FLPEWTGIA | 34 |
| RASGRP1 | HLLTLSLDL | 35 |
| RASGRP1 | KTAQDTLYV | 36 |
| IFIH1 | MMYKAIHCV | 37 |
| RASGRP1 | RLLDPGCLV | 38 |
| RASGRP1 | RMMVSLGHL | 39 |
| RASGRP1 | SLGMLSVLA | 40 |
| CYP27B1 | STPSFLAEL | 41 |
| IFIH1 | YLTFLPAEV | 42 |
| RASGRP1 | FSDYQNYLV | 43 |
| FUT2 | IMTIGTFGI | 44 |
| SH2B3 | RLSSYVVVV | 45 |
| RASGRP1 | VVVDWASGV | 46 |

TABLE 2
Exemplary T cell epitopes Associated with T1D

| | Epitope | SEQ ID |
|---|---|---|
| Antigen | sequence | NO |
| G6PC2 | VIGDWLNLI | 47 |
| CCNI | KLSPSQHLA | 48 |
| PCSK2 | KMAKDWKTV | 49 |
| CCNI | LQFRGSMLA | 50 |
| KIF1A | VLQASSISA | 51 |
| PTPRN2 | FSESILTYV | 52 |
| PTPRN2 | MVWESGCVV | 53 |
| PTPRN | FLPYDHARI | 54 |
| INS | ALMGQAAGV | 55 |
| INS | SVALWNNAV | 56 |
| KIF1A | NLLYPVPLV | 57 |
| UCN3 | FLLLLLLLL | 58 |
| KCNK16 | ILLGLAWLA | 59 |
| UCN3 | NLLFNIAKA | 60 |
| KCNK16 | VILIFPPMV | 61 |
| KIF1A | ALSSRAASV | 62 |
| KIF1A | AMVAALSPA | 63 |
| KIF1A | CVFLWPFLL | 64 |
| KIF1A | EMYDRAAEV | 65 |
| KIF1A | FLDSDIPSV | 66 |
| CCNI | FLHIFHAIA | 67 |
| PCSK2 | FMTDIIEAS | 68 |
| KIF1A | FQSESCPVV | 69 |
| GNAS | GIFETKFQV | 70 |
| GNAS | KVNFHMFDV | 71 |
| GNAS | LIDCAQYFL | 72 |
| PCSK2 | LMLHGTQSA | 73 |
| KIFIA | QMSGSTTTI | 74 |
| KIF1A | SLHERILFA | 75 |
| CCNI | SLLDRFLAT | 76 |
| KIF1A | TLGKVISAL | 77 |
| PCSK2 | TLQAMADGV | 78 |
| SCG5 | WLASGWTPA | 79 |
| PCSK2 | YLEHVQAVI | 80 |
| KIF1A | YLHFLEPHT | 81 |
| CCNI | YQFNLYPET | 82 |
| PCSK2 | GLDLNVAEA | 83 |
| KIF1A | YIKDGITRV | 84 |
| S100B | FMAFVAMVT | 85 |
| SLC3A8 | FSILLMEGV | 86 |
| SLC3A8 | SLAVVTDAA | 87 |
| PTPRN2 | LLPPRVLPA | 88 |
| PTPRN2 | RLLQVPSSA | 89 |
| PTPRN2 | TVADFWQMV | 90 |
| PTPRN2 | AMDFYRYEV | 91 |
| PTPRN2 | FLSWYDRGV | 92 |
| PTPRN2 | LMAGLMQGV | 93 |
| PTPRN2 | WQDDYTQYV | 94 |
| PTPRN | ASLYHVYEV | 95 |
| PTPRN | GVAGLLVAL | 96 |
| PTPRN | ILAYMEDHL | 97 |
| PTPRN | LLVALAVAL | 98 |
| PTPRN | LTLVALAGV | 99 |
| PTPRN | QLSTLLTLL | 100 |
| PTPRN | TVIVMLTPL | 101 |
| PTPRN | VLLEKKSPL | 102 |
| PTPRN | VLLTLVALA | 103 |
| SLC3A8 | LLMEGVPKS | 104 |
| PTPRN | RTLTQFHFL | 105 |
| PTPRN | SLADVTQQA | 106 |
| G6PC2 | WLIQISVCI | 107 |
| G6PC2 | YVLSFCKSA | 108 |
| GAD2 | ALGIGTDSV | 109 |
| GAD2 | FLIEEIERL | 110 |
| GAD2 | KQKGFVPFL | 111 |
| GAD2 | AMMIARFKM | 112 |
| INS | ALFHNATLL | 113 |
| INS | HMLHEPSHV | 114 |
| INS | RAEGCWAPA | 115 |
| INS | VVLQPAGPL | 116 |
| INS | WLDPRPQLC | 117 |
| INS | ALQVLCLPA | 118 |
| INS | HLQVLCLPA | 119 |
| INS | MLGPRWLYS | 120 |
| CCNI | NLYPETFAL | 121 |
| CCNI | QLLFSLPKL | 122 |
| GNAS | YMCTHRLLL | 123 |

TABLE 3
Exemplary T cell epitopes Associated with T1D

| | | SEQ |
|---|---|---|
| Antigen | Epitope sequence | ID NO |
| UCN3 | MLMPVHFLL | 124 |
| CCNI | KLNWDLHTA | 125 |
| KCNK16 | ALLGIPLNV | 126 |
| CHGA | KMDQLAKEL | 127 |
| GNAS | AMSNLVPPV | 128 |
| REXO2 | SVANALWIV | 129 |
| PubMed_27183389, | RQFGPDWIVA | 130 |
| Insulin mimotope | | |
| PubMed_27183389, | RQWGPDPAAV | 131 |
| Insulin mimotope | | |
| CHGA | TLSKPSPMPV | 132 |
| INS | IVEQCCTSI | 133 |
| INS | ALWGPDPAA | 134 |
| GAD2 | RMMEYGTTMV | 135 |
| SLC3A8 | SISVLISAL | 136 |
| SLC3A8 | TMHSLTIQM | 137 |
| G6PC2 | YLLLRVLNI | 138 |
| SLC3A8 | HIAGSLAVV | 139 |
| GAD2 | RFKMFPEVK | 140 |
| INS | IVEQCCTSI | 141 |

### Peptide or Nucleotide Compositions

The present disclosure provides peptides comprising one or more T cell epitopes set forth in Table 1, Table 2, and/or Table 3. Given the immunoprevalence of these T cell epitopes in T1D patients, the peptides comprising these T cell epitopes are recognized herein as T1D-associated peptides. The present disclosure also provides nucleotide sequences encoding one or more of such peptides or compositions (e.g., pharmaceutical compositions) comprising one or more of such peptides and the nucleotide sequences.

In certain embodiments, the present disclosure provides a peptide (e.g., isolated peptide) comprising a T cell epitope set forth in Table 1. In certain embodiments, the present disclosure provides a composition (e.g., pharmaceutical composition) comprising a peptide disclosed herein or a nucleotide sequence encoding the peptide. In certain embodiments, the composition comprises a plurality of peptides, a nucleotide sequence encoding the plurality of peptides, or a plurality of nucleotide sequences encoding the plurality of peptides, including at least one peptide comprising a T cell epitope set forth in Table 1.

In certain embodiments, the present disclosure provides a peptide (e.g., isolated peptide) comprising a T cell epitope set forth in Table 2 or a nucleotide sequence encoding the peptide. In certain embodiments, the present disclosure provides a composition (e.g., pharmaceutical composition) comprising a peptide disclosed herein or a nucleotide sequence encoding the peptide. In certain embodiments, the composition comprises a plurality of peptides, a nucleotide sequence encoding the plurality of peptides, or a plurality of nucleotide sequences encoding the plurality of peptides, including at least one peptide comprising a T cell epitope set forth in Table 2.

In certain embodiments, the present disclosure provides a peptide (e.g., isolated peptide) comprising a T cell epitope set forth in Table 3 or a nucleotide sequence encoding the peptide. In certain embodiments, the present disclosure provides a composition (e.g., pharmaceutical composition) comprising a peptide disclosed herein or a nucleotide sequence encoding the peptide. In certain embodiments, the composition comprises a plurality of peptides, a nucleotide sequence encoding the plurality of peptides, or a plurality of nucleotide sequences encoding the plurality of peptides, including at least one peptide comprising a T cell epitope set forth in Table 3.

In some aspects, the present disclosure provides means for inducing a tolerogenic immune response in a subject in need thereof in combination with a pharmaceutically acceptable carrier. In some aspects, the tolerogenic immune response is against one or more T cell epitopes disclosed herein.

Also contemplated are peptides comprising variants of the T cell epitopes. For example, a peptide can comprise an amino acid sequence that differs by 1, 2, or 3 amino acids (e.g., substitutions, insertions, or deletions) relative to a T cell epitope disclosed herein. In specific embodiments, a peptide can comprise an amino acid sequence of a T cell epitope set forth in Table 1, Table 2, or Table 3, with the N-terminal or C-terminal amino acid deleted. Such variants can be derived from, for example, mutant forms of the respective antigens that may be present in the human population. It is understood, according to scientific literature and databases (Rammensee et al., 1999 (supra); Godkin et al., 1997 (supra)), that certain positions of T cell epitopes (e.g., positions 2 and 9 of class I epitopes) are typically anchor residues forming a core sequence fitting to the binding groove of the MHC. Thus, a skilled person in the art would be able to modify the amino acid sequences of a T cell epitope disclosed herein, by maintaining the known anchor residues, and determine whether such variants maintain the ability to bind the MHC. Alternatively, the MHC anchor residues can be modified to improve binding of the peptide to the MHC, without affecting the remaining residues that interact with the cognate TCR. The peptides (e.g., isolated peptides) comprising the T cell epitopes disclosed herein or their variants are useful for stimulating T cell immune responses in vitro, ex vivo, or in vivo.

In certain embodiments, the peptide comprising a T cell epitope set forth in Table 1, Table 2, or Table 3, or a variant of such T cell epitope, is no more than 100 amino acids, no more than 90 amino acids, no more than 80 amino acids, no more than 70 amino acids, no more than 60 amino acids, no more than 50 amino acids, no more than 40 amino acids, no more than 30 amino acids, no more than 25 amino acids, no more than 20 amino acids, no more than 19 amino acids, no more than 18 amino acids, no more than 17 amino acids, no more than 16 amino acids, no more than 15 amino acids, no more than 14 amino acids, no more than 13 amino acids, no more than 12 amino acids, no more than 11 amino acids, no more than 10 amino acids, no more than 9 amino acids, or no more than 8 amino acids in length. In certain embodiments, the peptides does not comprise more than 100 (e.g., more than 90, more than 80, more than 70, more than 60, more than 50, more than 40, more than 30, more than 25, more than 20, more than 19, more than 18, more than 17, more than 16, more than 15, more than 14, more than 13, more than 12, more than 11, more than 10, more than 9, or more than 8) contiguous amino acids of an antigen set forth in Table 1, Table 2, or Table 3. In certain embodiments, the peptide comprises at least 20%, at least 30%, at least 40%, at least 50%, at least 60%, at least 70%, at least 80%, at least 90%, at least 95%, at least 99%, or 100% of the entire length of the corresponding antigen.

In certain embodiments, a peptide disclosed herein comprises two or more T cell epitopes of the same antigen. In certain embodiments, the two or more T cell epitopes are partially overlapping, and the peptide comprises the entire amino acid sequences of the two or more T cell epitopes aligned. In certain embodiments, the peptide comprises a T cell epitope presentable by a class I MHC and a T cell epitope presentable by a class II MHC. Without wishing to be bound by theory, it is contemplated that the T cell epitopes of such peptide can be presented by both class I and class II MHC proteins, thereby capable of stimulating an immune response by both CD8⁺ and CD4⁺ T cells.

A peptide comprising a T cell epitope may further comprise a moiety (e.g., an amino acid sequence) that improves one or more characteristics of the T cell epitope or its manufacture or function. For example, in certain embodiments, the peptide further comprises an amino acid sequence that facilitates delivery of the T cell epitope into an APC. In certain embodiments, the peptide comprises a cell penetrating peptide, which facilitates cell uptake in a manner that does not require a cell membrane protein. In certain embodiments, the peptide comprises a lipidation moiety, for example, to improve the half-live of the peptide. Exemplary cell penetrating peptides are disclosed in the CPPsite 2.0 database (crdd.osdd.net/raghava/cppsite/), which includes more than 1,000 unique cell penetrating peptides. In certain embodiments, the peptide further comprises a binding moiety (e.g., an antibody or an antigen-binding fragment thereof) that targets an APC (e.g., professional APC). Exemplary targets of such binding moiety include CD11b, CD11c, CD18, CD24, CD1a, CD206, DC inhibitory receptor 2 (DCIR2), and DEC205. In certain embodiments, the peptide further comprises a moiety that improves immunogenicity of the T cell epitope. For example, in certain embodiments, the peptide further comprises a heat shock protein-binding motif (see WO2019210055A2). Peptides can be synthesized by the translation machinery (optionally in combination of transcription machinery) of a cell either in the cell or in vitro. Alternatively, peptides can be chemically synthesized. In certain embodiments, the peptide is modified or comprises a non-germline encoded peptide, for example, to comprise or mimic a post-translational modification (PTM) of the antigen protein when expressed in an APC. Modified peptides comprise, but are not limited, to post-translational modifications (PTMs) and other non-germline encoded peptides as reviewed in (Mannering et al., (2019) Diabetologia 62:351-356, Doyle et al., (2014) Autoimmunity 47:220-233), such as defective ribosomal products (Kracht et al., (2017) Nat Med 23:501-507), peptide fusions (Babon et al., (2016) Nat Med 22:1482-1487, Delong et al., (2016) Science 351:711-714), amino acid substitutions or changes such as arginine to citrulline or deamidation (Babon et al., (2016) Nat Med 22:1482-1487, Mclaughlin et al., (2016) Clin Exp Immunol 185:133-140) that have been shown to be antigenic epitopes in Type I diabetes or other T cell-based studies. Other modified peptides include vicinal disulfide bond (Mannering et al., (2005) J Exp Med 202:1191-1197), glutamine to glutamic acid conversion (Lummel et al., (2014) Diabetes 63:237-247), arginine to citrulline (Babon et al., (2016) Nat Med 22:1482-1487, Azoury et al., (2021) Diabetes 70:2879-2891), chlorination (Sidney et al., (2018) BMC Immunol 19:12), oxidation (Sidney et al., (2018) BMC Immunol 19:12), deamidation (Sidney et al., (2018) BMC Immunol 19:12), alternative splicing (Jong et al., (2013) Diabetologia 56:2651-2658), carbonylation (Yang et al., (2017) Antioxid Redox Signal 29:1415-1431), aspartic acid to isoaspartic acid (Doyle et al., (2013) Autoimmunity 46:6-13), glycosylation (Mei et al., (2020) Mol Cell Proteomics 19:1236-1247), or phosphorylation (Cobbold et al., Sci Transl Med. (2013) 5(203): 203ra125).

The present disclosure provides a composition (e.g., pharmaceutical composition) comprising a plurality of different peptides disclosed herein. In certain embodiments, the composition comprises two or more, three or more, four or more, five or more, six or more, seven or more, eight or more, nine or more, ten or more, 15 or more, 20 or more, 30 or more, 40 or more, 50 or more, 60 or more, 70 or more, 80 or more, 90 or more, 100 or more, 150 or more, 200 or more, or 300 or more different peptides comprising T cell epitopes disclosed herein. In certain embodiments, the composition comprises 5-500, 5-400, 5-300, 5-200, 5-150, 5-100, 5-90, 5-80, 5-70, 5-60, 5-50, 5-40, 5-30, 5-20, 5-10, 10-500, 10-400, 10-300, 10-200, 10-150, 10-100, 10-90, 10-80, 10-70, 10-60, 10-50, 10-40, 10-30, 10-20, 20-500, 20-400, 20-300, 20-200, 20-150, 20-100, 20-90, 20-80, 20-70, 20-60, 20-50, 20-40, 20-30, 30-500, 30-400, 30-300, 30-200, 30-150, 30-100, 30-90, 30-80, 30-70, 30-60, 30-50, 30-40, 40-500, 40-400, 40-300, 40-200, 40-150, 40-100, 40-90, 40-80, 40-70, 40-60, 40-50, 50-500, 50-400, 50-300, 50-200, 50-150, 50-100, 50-90, 50-80, 50-70, 50-60, 100-500, 100-400, 100-300, 100-200, 100-150, 150-500, 150-400, 150-300, 150-200, 200-500, 200-400, or 200-300 different peptides comprising T cell epitopes disclosed herein. In certain embodiments, the composition comprises a first peptide comprising a T cell epitope presentable by a class I MHC and a second peptide comprising a T cell epitope presentable by a class II MHC, optionally wherein the first and second peptides are different. Without wishing to be bound by theory, it is contemplated that the T cell epitopes in such composition can be presented by both class I and class II MHC proteins, thereby capable of stimulating an immune response by both CD8⁺ and CD4⁺ T cells.

In certain embodiments, a composition of peptide(s) disclosed herein, when presented by cognate MHC(s), is capable of stimulating a T cell response, for example, enhancing survival, proliferation, activation, and/or memory formation of one or more T cells, in a T cell population obtained from a subject (e.g., peripheral blood mononuclear cells from a human subject) under ex vivo or in vivo conditions. Where the cognate MHC is a class I MHC, the peptide is capable of stimulating a CD8⁺ T cell response. The peptide-MHC complex can be a soluble complex, immortalized on a solid surface (e.g., bead or nanoparticle), or presented on the surface of an APC. Viability of T cells can be assessed by methods known in the art such as cell counting or dye exclusion assays (e.g., with trypan blue or propidium iodide). Clonal T cell proliferation can be assessed by methods known in the art such as carboxyfluorescein succinimidyl ester (CFSE) dilution assay, Ki-67 staining, tritiated thymidine incorporation, BrdU incorporation (see Gratzner, Science (1982) 218(4571): 474-75), or quantitation of ATP levels (e.g., with CellTiter-Glo Cell Viability assay). T cell activation can be assessed by methods known in the art such as staining for cell surface markers (e.g., upregulation of CD69, CD27, CD137, CD154, CD25, CD44, or CD107a, or downregulation of CD62L or CCR7) or cytokines (e.g., IFNγ or TNFα) or detecting secretion of cytokine proteins (e.g., IFNγ or TNFα) or proteins associated with cytolytic activity (e.g., GranzymeB or Perforin). Memory T cell formation can be assessed by methods known in the art such as staining for cell surface markers (e.g., CD45RO, CD95, Scal, CD62L, CD27, CD127, and CD44). In certain embodiments, the composition is capable of enhancing survival, proliferation, activation, and/or memory formation of both CD8⁺ and CD4⁺ T cells.

Soluble pMHC Complexes

The present disclosure also provides a protein complex comprising a peptide disclosed herein complexed with a cognate MHC (e.g., HLA-A*02:01, e.g., a soluble HLA-A*02:01). Where the MHC is a class I MHC (e.g., HLA-A*02:01), in certain embodiments, the protein complex comprises α1 and α2 domains of the class I MHC. In certain embodiments, the protein complex further comprises α3 domain and β2-microglobulin (β2m) of the class I MHC.

In certain embodiments, the peptide is fused with at least one polypeptide of the class I MHC (e.g., HLA-A*02:01). For example, a single-chain construct can be generated from a peptide and cognate class I MHC, wherein the C-terminus of the peptide is linked to the N-terminus of β2m by a first flexible linker and the C-terminus of β2m is linked to the N-terminus of the MHC heavy chain (comprising α1, α2, and α3 domains) by a second flexible linker (see Hansen et al., Trends Immunol. (2010) 31(10): 363-69). In certain embodiments, the first and second flexible linkers are peptide linkers, such that the single-chain construct can be produced recombinantly.

In certain embodiments, the peptide and/or the MHC (e.g., as a single-chain construct) is fused with a multimerization domain so as to form a multimer (e.g., dimer, trimer, tetramer, pentamer, or hexamer). Without wishing to be bound by theory, it is contemplated that the multimerization increases the avidity of the pMHC, thereby to compensate for the often low affinity of pMHCs to cognate TCRs. In addition to targeting T cells in vivo, such multimer can be used to probe T cells or isolated TCRs to identify a TCR that binds the T cell epitope. In certain embodiments, the multimerization domain comprises avidin or a variant thereof (e.g., streptavidin). In certain embodiments, the complex further comprises a barcode, for example, a barcode nucleic acid conjugated with a biotin that binds a streptavidin that is fused with the peptide and/or MHC. A pMHC multimer can comprise a plurality of the same pMHC. Alternatively, it can comprise a plurality of different pMHCs, e.g., two or more copies of a first pMHC and two or more copies of a second, different pMHC. In certain embodiments, the multimerization domain comprises an antibody (e.g., IgG, IgM, IgA) Fc region or a fragment thereof that mediates multimerization. In certain embodiments, the multimerization domain is a dimerization domain comprising an IgG (e.g., IgG4) Fc region or a fragment thereof that mediates dimerization (e.g., CH3 domain) (see Suarez et al., Transfus. Med. Hemother. (2020) 47:464-71). In certain embodiments, the multimerization domain comprises an IgM Cμ4 constant region or IgA Cα3 constant region, and optionally further comprises a corresponding tailpiece region (see, e.g., International Application No. PCT/US2022/023898). In certain embodiments, the multimerization domain comprises a Cμ2, Cμ3, and Cμ4 domain of IgM. In certain embodiments, the multimer further comprises an IgM tailpiece region. In certain embodiments, the multimer further comprises a J-domain, optionally wherein the J-domain can be fused with an immunomodulator. Exemplary immunomodulators are described in the “Presentation Agents” subsection below. In certain embodiments, the immunomodulator comprises a PD-1 agonist, for example, an extracellular fragment of PD-L1 or an anti-PD-1 agonistic antibody or an antigen-binding fragment thereof.

In certain embodiments, the soluble pMHC (e.g., a single-chain construct) is linked to (e.g., fused with) an effector that reduces survival, proliferation, activation, and/or memory formation of one or more cytotoxic T cells. The soluble pMHC targets the effector to T cells that bind the pMHC, thereby specifically directing the effector's function to such T cells. Non-limiting examples of suitable effectors include checkpoint protein agonists, anti-inflammatory cytokines, and cell death-inducing agents. For example, in some embodiments, the effector comprises PD-L1 (e.g., Domain 1 or full extracellular domain), an agonistic antibody that binds PD-1, TIGIT, CTLA4, TIM-3, LAG-3, LILRB1, or LILRB2, or a binding domain or portion (e.g., an antigen-binding fragment) of any of these antibodies. In some embodiments, the effector comprises a TNF receptor superfamily agonist (e.g., a TNF family molecule), such as CD137L, an anti-CD137 agonistic antibody, or an antigen-binding fragment thereof. In some embodiments, the effector comprises HLA-G or an extracellular fragment thereof. In some embodiments, the effector comprises an anti-inflammatory cytokine such as TGFβ or IL-10, which promotes Treg phenotype and context-dependent immunosuppression effects. In some embodiments, the effector comprises an inducer of cell death. For example, in some embodiments, the inducer of cell death is a TNF receptor superfamily agonist, such as FasL, an anti-Fas agonistic antibody or an antigen-binding fragment thereof, TRAIL, an agonistic anti-DR4 or anti-DR5 antibody or an antigen-binding fragment thereof. In some embodiments, the inducer of cell death is a cytotoxic agent such as a microtubule inhibitor or DNA damaging agent. It is contemplated that two or more such effector moieties, either separately or conjugated or fused together, may be linked to the soluble pMHC.

### TCR Mimetics

In some aspects, binding moieties that mimic TCRs, referred to as TCR-mimetic antibodies or TCR-like antibodies (TCRL), reactive to or specifically bind to the epitopes in the context of HLA-A*0201 listed in Tables 1-3 could be used for therapeutic benefit in T1D and autoimmune disease in general as outlined in Li et al., (2022) Front. Immunol 13-2022 (PMID: 35967436). Briefly, TCRLs would have the ability to overcome limitations of targeting immunotherapies based on pMHC-TCR identification in autoimmune context, namely the relatively low-affinity of autoreactive pMHC-TCR interactions. By overcoming these low-affinities, one could use the disclosure listed to create TCRLs to one or more epitopes, Tables 1-3, in order to drive TCRL-directed depletion or modulation of specific immune populations, such as antigen presenting cells, in order to drive therapeutic benefit in T1D and autoimmune disease in general. Some examples of using TCRL-directed depletion include but are not limited to; Fc-independent antibody-induced apoptosis, Fc-mediated effector responses, depleting bispecific antibodies, toxin-induced depletion, and target cell depletion via TCRL CAR-T cells. Some examples of using TCRL-modulation include but are not limited to: self-epitope blockade, cytokine induced mediation, non-depleting bispecific molecules, and tolerance induction via TCRL CAR Tregs.

### III. Antigen Presenting Cells

In another aspect, the present disclosure provides an APC composition comprising one or more antigen-presenting cells (APCs) presenting or capable of presenting one or more T cell epitopes disclosed herein. In certain embodiments, the APC is a tolerogenic APC.

### APCs and Tolerogenic APCs

The present disclosure provides tolerogenic antigen-presenting cells (APCs) or a population or composition (e.g., pharmaceutical composition) thereof. APCs are a group of cells (e.g., immune cells) that mediate the cellular immune response by displaying endogenous and exogenous antigen complexed to major histocompatibility complexes (MHCs) class I and class II (“MHC I” and “MHC II”) on the cell surface for recognition by certain lymphocytes such as T cells. Tolerogenic APCs can be found in all tissues but are found in abundance in the skin, liver, spleen, and lymph nodes. In certain embodiments, the tolerogenic APC expresses scavenger receptor MARCO, ILT-3, ILT-4, and/or CD52.

APCs include professional APCs and non-professional APCs. Professional APCs include but are not limited to dendritic cells (DCs), macrophages, Langerhans cells, and B cells. Non-professional APCs include but are not limited to endothelial cells, fibroblasts, certain cells in the liver, and lymph node stromal cells that are capable of expressing MHC I and MHC II proteins and can present exogenous antigens. An APC can also be an artificial APC (e.g., drosophila cells) engineered to express a MHC that presents a T cell epitope. Also contemplated herein are artificial APCs, for example, a bead, nanoparticle, or liposome, that presents a peptide-MHC on the surface. artificial APCs can also be used to prime or stimulate T cells ex vivo or in vivo.

A function of dendritic cells (DCs) is to present antigens to T cells. Dendritic cells typically use two types of major histocompatibility complex (MHC) to display antigen peptides: MHC I and MHC II. MHC I trains CD8+ T-cells into cytotoxic, tumor-cell killers; and MHC II trains CD4+ T-cells into cytokine-producing helper cells. Clinical and pre-clinical data suggest that both T-cell types help to kill tumors. The peptides presented by MHC I and MHC II are not necessarily the same between each other, or between patients.

Immature dendritic cells (iDCs) are characterized by high endocytic activity and low T-cell activation potential. Immature dendritic cells phagocytose pathogens and degrade their proteins into small pieces and upon maturation present those fragments at their cell surface using MHC molecules. Simultaneously, iDCs upregulate cell-surface receptors that act as co-receptors in T-cell activation such as CD80 (β7.1), CD86 (β7.2), and CD40, greatly enhancing their ability to activate T-cells. Once iDCs have come into contact with a presentable antigen, iDCs become activated and mature into mature dendritic cells (mDCs), which in turn, can activate helper T-cells and cytotoxic T-cells as well as B-cells by presenting them with antigens derived from the pathogen, alongside non-antigen specific costimulatory signals.

Tolerogenic DCs (tolDCs) comprise several subsets of cells, for example VitD3-tolDC, Rapa-DC, DC10, Dex-tolDC, and ATDC. TolDCs induce tolerance via several contact-dependent and contact-independent mechanisms. TolDCs are generally characterized by low expression of costimulatory molecules (CD80, CD86, and CD83), upregulation of inhibitory and modulatory receptors, secretion of low pro-inflammatory and high levels of anti-inflammatory cytokines. TolDCs present antigens through normal T cell TCR and DC MHC interactions. However, mature tolerogenic DCs express high levels of CD86, which stimulates CD28 signaling to stabilize Treg cells (see Halliday et al., Front. Immunol. (2020) 11:600000). In certain embodiments, a tolDC is generated by loading or pulsing an immature dendritic cell (iDC) in vitro with apoptotic bodies from a cell that expresses an autoantigen and/or presents a T cell epitope thereof (e.g., one or more T cell epitopes disclosed herein) (see Marin-Gallen et al., Clin. Exp. Immunol. (2010) 160(2): 207-14). In certain embodiments, a tolDC is generated by loading or pulsing an iDC in vitro with one or more T cell epitopes of an autoantigen (e.g., one or more T cell epitopes disclosed herein) and culturing the iDC under immunosuppressive conditions. Exemplary immunosuppressive conditions include but are not limited to presence of an agonist of an immune checkpoint protein (e.g., an agonistic anti-CTLA-4 antibody or an antigen-binding fragment thereof conjugated to an anti-CD11c antibody or an antigen-binding fragment thereof for coating the iDC). Interaction tolDCs with T cells leads to inhibition of effector T cell responses and the induction of regulatory T cells (Tregs).

In certain embodiments, the target cell is a tolerogenic target cell in the spleen, such as a marginal zone macrophage, a metallophilic macrophage, or a marginal zone dendritic cell. Marginal zone dendritic cells (Langerin CD8⁺ DC) reside in the splenic marginal zone and are important for the regulation of tolerance toward cell-associated self-antigens. Markers for marginal zone DCs include CD8 and CD103.

In certain embodiments, dendritic cells can be generated in vivo or ex vivo from monocytes, sometimes referred to as monocyte-derived dendritic cells. Briefly, monocytes can transition from CD14+CD83-monocytes to CD14-CD83-immature DCs under the influence of IL-4 and GMCSF and then upregulate CD83 upon activation/maturation to become CD14-CD83+mDC (see, e.g., Putz et al., Methods Mol Med. (2005) 109:71-82). In general, the transition from immature to mature DC is not instantaneous and requires some time, during which time the DCs undergo a maturing process. Some DCs may mature faster than others and thus, the population may be a mix of immature, maturing, semi-mature, and mature DCs, while the population as a whole is in the process of maturing.

Monocyte-derived dendritic cells (moDC) can also be generated in vitro from peripheral blood mononuclear cells (PBMCs). Plating of PBMCs in a tissue culture flask permits adherence of monocytes. Treatment of these monocytes with interleukin 4 (IL-4) and granulocyte-macrophage colony stimulating factor (GM-CSF) leads to differentiation to immature dendritic cells. Subsequent treatment with tumor necrosis factor (TNF), IL-6, IL-1B, and/or PGE2 further differentiates the iDCs into mature DCs. Marcrophages are effector cells of the innate immune system that phagocytose bacteria and secrete both pro-inflammatory and antimicrobial mediators. In addition, macrophages play an important role in eliminating diseased and damaged cells through their programmed cell death. There are three main groups of macrophages, classically-activated (M1) macrophages, wound-healing macrophages (also known as alternatively-activated (M2) macrophages), and regulatory macrophages (Mregs). Regulatory macrophages (Mregs) can provide a tolerogenic environment. Marginal zone macrophages (MZM or MZMϕ) and metallophilic macrophages (MMMϕ) are a subset of specialized splenic macrophages that interact with apoptotic material entering the spleen from circulation. Hepatic macrophages comprise a heterogeneous population of cells, for example, Kupffer cells (KCs), and recruited macrophages derived from peritoneal cavity as well as the bone marrow. Hepatic macrophages can be tolerogenic antigen-presenting cells that inhibit T-cell activation by producing distinct sets of cytokines, chemokines, and mediators to maintain or resolve inflammation. Markers for macrophages include CD11b/Integrin alpha M, CD14, CD68, Fc gamma RIII/CD16, Fc gamma RI/CD64, and CCR5.

In certain embodiments, the target cell is a tolerogenic target cell in the liver, such as a liver sinusoidal endothelial cell (LSEC), MARCO⁺ Kupffer cell, or a monocyte-derived macrophage. LSECs are in direct contact with blood and possess efficient endocytic pathways for exogenic antigen endocytosis. Markers for LSECs include CD31, CD36, stabilin 1, 2, and B1, lipoprotein receptor-related protein-1, and certain C-type lectin receptors. For example, Type 1 LSEC are CD36hiCD32⁻CD14⁻LYVE-1⁻, while Type 2 LSEC are LYVE-1⁺CD32hiCD14⁺CD54⁺CD36mid-lo. LSECs are not only able to present exogenous antigen on MHC II but also on MHC I and are capable of antigen cross-presentation to CD8⁺ T cells. LSECs can upregulate the co-inhibitory molecule β7-H1 in CD8⁺ T-cells, and shift the balance from activation to tolerance induction in CD8⁺ T-cells. Naïve CD4⁺ T-cells primed by LSECs under steady state can differentiate into regulatory T-cells (Tregs) that lack the transcription factor Forkhead-Box-Protein P3 (FoxP3), which is normally expressed by Tregs generated by professional APCs. LSEC-induced CD25^{low}FoxP3⁻ T-cells are immune suppressive.

Hepatocytes are liver cells that can present exogenic antigens on MHC I and, in inflammatory conditions, MHC II molecules. Recently, hepatocytes were shown to be capable of priming naïve CD8⁺ T-cells directly and via cross-presentation and of activating CD4⁺ T-cells leading to the generation of CD4⁺CD25⁺FoxP3⁺ Tregs (reviewed in Mehrfeld et al., Front. Immunol. (2018) 9:635). Markers for hepatocytes include KRT8 and KRT18.

Lymph node stromal cells (LNSC) are a group of cells that include, for example, fibroblastic reticular cells, and lymphatic and blood endothelial cells. LNSCs can promote a tolerogenic CD4+ T cell compartment and can aid in the deletion of antigen-specific CD8+ T cells. Markers for LNSCs comprise podoplanin (gp38) and PECAM-1 (CD31). Lymphatic endothelial cells have a high endocytic capacity, are capable of presenting exogenous antigens to T-cells on both MHC-I and MHC-II molecules, and mediate the deletion of autoreactive CD8+ T-cells.

In some embodiments, the APC is an artificial APC, for example, a cell line engineered to express a MHC (see Turtle et al., Cancer J. (2010) 16(4): 374-81). In some embodiments, the cell line is a lymphoblast cell line (e.g., K562). aAPCs are designed to mimic the temporal and spatial aspects of T cell-APC interactions. Such artificial APCs can be used to activate T cells (e.g., Treg cells) that express a cognate TCR in vitro or in vivo.

### Peptide Loading

In certain embodiments, the APC composition disclosed herein is prepared by loading cells (e.g., monocytes and/or dendritic cells) with a peptide composition disclosed herein (see the “Peptide Compositions” subsection of Section II above), using the preloading method and/or the direct loading method.

Methods for peptide loading on APCs are known in the art, for example as described in WO2020/055931. Briefly, APCs such as monocytes, iDCs, and other non-mature DCs can be loaded with antigen peptides for presenting the incorporated T cell epitopes on the cell surface. In a “preloading” method, monocytes and/or immature DCs are induced to internalize and proteolytically process the antigen peptides into shorter fragments (T cell epitopes) for subsequent loading onto class I and class II MHCs and presentation to T cells. The processed antigen peptides may be stored by the monocytes and/or immature DCs during the differentiation and/or maturation process and subsequently presented on the surface of mature DCs. In certain embodiments, monocytes are incubated in the presence of one or more peptides prior to and/or during differentiation. In certain embodiments, immature DCs are incubated in the presence of one or more peptides prior to and/or during maturation. Alternatively, in a “direct loading” method, peptides can be directly loaded onto mature DCs. These peptides are directly loaded on the cell surface MHCs and are generally not shortened by intracellular processing. In certain embodiments, preloading of immature DCs and direct loading of mature DCs can be combined, resulting in a mixed population of pre-loaded and directly loaded mature DCs.

Alternatively, the APC composition disclosed herein can be prepared by nucleic acid transfection. For example, in certain embodiments, the APCs are prepared by transfecting a population of cells ex vivo with one or more nucleic acids encoding an antigen or a portion thereof (e.g., at least 50%, at least 60%, at least 70%, at least 80%, at least 90%, at least 95%, or 100% of the antigen). Nucleic acids useful herein include DNA and RNA (e.g., mRNA), including modified forms thereof. In certain embodiments, the nucleic acids transfected comprise mRNA, optionally mRNA having one or more chemical modifications. Nucleic acid compositions described in the “Tolerogenic Peptide Compositions” subsection below are also contemplated for use in this ex vivo transfection method.

In certain embodiments, the APCs of the APC composition are derived from a single subject and thus, as a population, express up to two variants from the two alleles of each MHC genetic locus (e.g., HLA-A, HLA-B, HLA-C, HLA-DP, HLA-DQ, HLA-DR). In other embodiments, the APCs of the APC composition are pooled from a plurality of subjects and thus have greater MHC diversity.

In certain embodiments, the APCs of the APC composition present one or more T cell epitopes disclosed herein. In certain embodiments, the APCs (e.g., APCs derived from a single subject) present a plurality of different T cell epitopes disclosed herein. In certain embodiments, the APCs (e.g., APCs derived from a single subject) present two or more, three or more, four or more, five or more, six or more, seven or more, eight or more, nine or more, ten or more, 15 or more, 20 or more, 30 or more, 40 or more, 50 or more, 60 or more, 70 or more, 80 or more, 90 or more, or 100 or more different T cell epitopes disclosed herein. In certain embodiments, the APCs (e.g., APCs derived from a single subject) present 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 different T cell epitopes disclosed herein. In certain embodiments, the APCs present a first T cell epitope by a class I MHC and a second T cell epitope by a class II MHC. Without wishing to be bound by theory, it is contemplated that such APCs are capable of stimulating an immune response by both CD8⁺ and CD4⁺ T cells.

In certain embodiments, at least 30, at least 40%, at least 50%, at least 60%, at least 70%, at least 80%, at least 90%, at least 95%, or at least 99% of the APCs in the APC composition present one or more T cell epitopes disclosed herein. In certain embodiments, the percentage of lymphocytes (e.g., T cells) relative to all cells in the APC composition is 20% or less, 10% or less, 5% or less, 4% or less, 3% or less, 2% or less, or 1% or less. In certain embodiments, the APC composition is substantially free of lymphocytes (e.g., T cells). In certain embodiments, lymphocytes (e.g., T cells) are removed from the APC composition, for example, by surface marker-based magnetic bead selection or cell sorting.

In certain embodiments, the peptide composition comprises an immunomodulator disclosed herein. It is contemplated that the immunomodulator may induce or stabilize a tolerogenic phenotype of the APC.

### IV. T Cells

In another aspect, the present disclosure provides a T cell composition comprising one or more T cells (e.g., regulatory T cells) that recognize one or more T cell epitopes presented by HLA-A*02:01 on an APC (e.g., artificial APC).

It is understood that T cell activation (priming) and expansion requires two signals. TCR engagement with an MHC presenting a cognate peptide is the first step and is termed “signal 1.” After initial activation by signal 1, T cells can be expanded by providing “signal 2,” which can be provided by, for example, the β7 family proteins β7.1 and β7.2 which interact with CD28, or the tumor necrosis factor receptor (TNFR) family ligands OX-40L, CD70, 4-1BBL, which interact with OX-40, CD27, and 4-1BB on the T cell, respectively. Certain signal 2 interactions can be inhibitory to T cell expansion and effector function, such as the interaction of CTLA-4 with β7.1 or PD-1 with PD-L1 (β7.H1) or PD-L2 (β7-DC). Methods for T cell priming and expansion are known in the art, e.g., co-culturing the lymphocyte-rich fraction of the PBMCs with the APCs disclosed herein to expand T cells that are reactive to the T cell epitopes disclosed herein. In some embodiments, the APCs loaded with T cell epitopes disclosed herein and prepared in accordance with the methods disclosed herein can be used to prime and expand certain T cells populations in vitro, ex vivo, and in vivo.

### CD8⁺ Regulatory T Cells

T cells are characterized by the expression of certain cell surface molecules such as the T cell receptor (TCR), a multiprotein complex that is involved in MHC binding. In some embodiments, the T cells are derived from blood, bone marrow, lymphoid organs (e.g., lymph nodes or spleen), or tumor biopsies from a subject. These cells typically are primary cells, such as those isolated directly from a subject and/or tissue isolated from a subject. Other exemplary sources of T cells include T cell derived from stem cells, such as multipotent and pluripotent stem cells, including induced pluripotent stem cells (iPSCs). Stem cells can be directly isolated from a subject such as from cord blood, blood, or tissue. iPSCs can be derived from any cell type that can be used to produce iPSCs. iPSCs can also be derived from a cell bank, and used to generate an allogeneic T cell product.

The T cells disclosed herein can be from a T cell subpopulation defined by function, activation state, maturity, potential for differentiation, expansion, recirculation, localization, and/or persistence capacities, antigen specificity, type of antigen receptor, presence in a particular organ or compartment, marker or cytokine secretion profile, and/or degree of differentiation. Included within this subpopulation of T cells are, for example, CD4⁺ T cells and CD8⁺ T cells.

Regulatory T (Treg) cells are a specialized subpopulation of T cells that act to suppress immune response, thereby maintaining homeostasis and self-tolerance. Tregs are able to inhibit T cell proliferation and cytokine production and play a critical role in preventing autoimmunity. Treg cells can be CD4⁺ Treg cells or CD8⁺ Treg cells.

CD8⁺ Treg cells possess important immunosuppressive functions. They can target antigen-activated CD4⁺ helper T cells with their cytotoxic activity, thereby suppressing the activity of the CD4⁺ helper T cells. Further, CD8⁺ Treg cells can function by secreting various inhibitory cytokines and chemokines, including IL-10, transforming growth factor (TGF)-β, IL-16, IFN-γ and chemokine (C-C motif) ligand 4, that can render APCs tolerogenic. Depending on the history of a cell, subpopulations of CD8⁺ Treg cells are characterized by several surface markers. In humans, the most prevalent are CD8 CD28⁺ Treg cells that exhibit age-dependent accumulation in human and sub-populations of CD8⁺CD122⁺CD49d⁺ T reg cells that express both PD-1 and IL-10 and suppress alloantigen-induced transplant rejections. CD8⁺ Treg cells can be primed and/or expanded by induction of CD8⁺ T cells, preferably naïve CD8+ T cells or natural regulatory CD8+ T cells, in vitro. Conditions for priming and/or expanding CD8⁺ Treg cells include but are not limited to TGF-β and IL-2, or IL-15 and IL-2, in the presence of APCs or anti-CD3/CD28 antibodies. Tolerogenic small molecules, such as rapamycin, cyclosporin A, methylprednisolone, or tacrolimus, can also be included in the cell culture for priming and/or expanding CD8⁺ Treg cells.

In certain embodiments, the present disclosure provides a population of T cells that recognize one or more T cell epitopes disclosed herein. In some embodiments, the T cells include one or more subsets of T cells, such as a whole T cell population, CD8⁺ cells, CD4⁺ cells, or subpopulations and combinations thereof.

In certain embodiments, the T cell composition comprises CD8⁺ T cells (e.g., CD8⁺ Treg cells). In certain embodiments, at least 30%, at least 40%, at least 50%, at least 60%, at least 70%, at least 80%, or at least 90% of the cells in the T cell composition are CD8⁺ T cells (e.g., CD8⁺ Treg cells). In some embodiments, the disclosure provides for a mixture of T cells, such as a mix of CD8⁺ and CD4⁺ cells. In certain embodiments in a mixture of CD8⁺ and CD4⁺ cells, the ratio of CD8⁺ to CD4⁺ cells can be, for example, 1:1, 1:2 to 2:1, 1:3 to 3:1, 1:4 to 4:1, 1:5 to 5:1, etc. Methods to measure the ratio of T cells are known in the art. For example, a ratio of CD8⁺ to CD4⁺ can be determined by staining the CD8⁺ and CD4⁺ cells with CD8 and CD4 specific antibodies and characterize and enumerate the cells for example by flow cytometric analysis. In certain embodiments, the T cell composition comprises alpha/beta T cells, and gamma/delta T cells. In some embodiments, the at least 30%, at least 40%, at least 50%, at least 60%, at least 70%, at least 80%, at least 90%, at least 95% of the cells in the T cell composition are alpha/beta T cells. In some embodiments, the at least 1%, at least 2%, at least 3%, at least 4%, at least 5%, at least 6%, at least 7%, at least 8%, at least 9%, or at least 10% of the cells in the T cell composition are gamma/delta T cells. For example, a ratio of alpha/beta T cells, and gamma/delta T cells can be determined by staining the alpha/beta T cells and gamma/delta T cells with alpha/beta and gamma/delta specific antibodies and characterize and enumerate the cells for example with flow cytometry.

The T cells can be harvested from blood, bone marrow, lymphoid organs (e.g., lymph nodes), or tumor biopsies from a subject. In some embodiments the T cells are harvested from a cell culture. The T cells can be purified before further culturing and expansion or can be used in a mixture with other cells. In certain embodiments, the primary T cells and stem cell derived T cells can be used fresh or frozen. The T cells can be maintained in ex vivo culture in the presence of one or more cytokines, such as IL-15 and/or IL-12, and optionally one or more of IL-21, IL-7, IL-2, IL-10, and IL-6.

With reference to the subject to be treated, the T cells may be allogeneic and/or autologous. Allogeneic T cells are suitable for use in off-the-shelf methods. In certain embodiments, an off-the-shelf method employs pluripotent and/or multipotent cells, such as stem cells and induced pluripotent stem cells (iPSCs), for producing T cells. In some embodiments, the present disclosure provides an autologous method that includes isolating cells from a subject, preparing, processing, culturing, and/or engineering the cells as described herein to produce T cells, and administering the T cells to the patient, with or without cryopreservation prior to the administration. In some embodiments, the T cells are autologous T cells. In some embodiments, the T cells are allogeneic T cells (e.g., cells from a healthy donor).

Also provided herein are populations of T cells reactive to the T cell epitopes disclosed herein and compositions containing such T cells. In some embodiments, a composition or population disclosed herein is enriched for such T cells. For example, in certain embodiments, at least 1%, 5%, 10%, 20%, 30%, 40%, 50%, 60%, 70%, 80%, 90%, 91%, 92%, 93%, 94%, 95%, 96%, 97%, 98%, or 99% of the total cells in the composition or population recognize one or more T cell epitopes disclosed herein. In certain embodiments, at least 1%, 5%, 10%, 20%, 30%, 40%, 50%, 60%, 70%, 80%, 90%, 91%, 92%, 93%, 94%, 95%, 96%, 97%, 98%, or 99% of the CD8+ cells in the composition or population recognize one or more T cell epitopes disclosed herein. In certain embodiments, at least 1%, 5%, 10%, 20%, 30%, 40%, 50%, 60%, 70%, 80%, 90%, 91%, 92%, 93%, 94%, 95%, 96%, 97%, 98%, or 99% of the CD4+ cells in the composition or population recognize one or more T cell epitopes disclosed herein.

Also provided herein are cell compositions comprising the T cells disclosed herein. In certain embodiments, the cell composition has been enriched for T cells (e.g., Treg cells). The enrichment can produce a composition in which at least 70%, at least 80%, at least 90%, at least 95%, or at least 99% of the cells in the composition are T cells (e.g., Treg cells). In certain embodiments, the composition is substantially free of myeloid cells. For example, in certain embodiments, the percentage of myeloid cells relative to all cells in the composition is 20% or less, 10% or less, 5% or less, 4% or less, 3% or less, 2% or less, or 1% or less. In certain embodiments, the cell composition has been enriched for T cells (e.g., Treg cells) reactive to one or more antigens of interest. Where the T cells are prepared by stimulation using APCs in an ex vivo cell culture, the T cells reactive to the epitopes presented by the APCs outgrow other T cells in the beginning cell culture. Given that the APCs can be generated such that they present, predominantly, epitopes of selected antigens, the resulting T cells can have a high reactive rate to the selected antigens. In certain embodiments, at least 50%, at least 60%, at least 70%, at least 80%, at least 90%, at least 95%, or at least 99% of the T cells in the population are reactive to one or more T cell epitopes of the selected antigens.

### T Cell Priming

In certain embodiments, the T cells (e.g., CD8⁺ Treg cells) disclosed herein express endogenous TCRs that bind one or more T cell epitopes disclosed herein. The T cells can be prepared by priming with an APC presenting the T cell epitopes (e.g., an APC composition disclosed herein in Section III) ex vivo or in vivo.

T cells can be obtained from a blood sample of a subject (e.g., a patient) by apheresis. In some embodiments, a lymphocyte-rich fraction and a monocyte-rich fraction can be acquired by elutriating peripheral blood mononuclear cells (PBMCs) of the subject. The monocyte-rich fraction can then be used to prepare antigen-presenting cells (APCs) for priming T cells, which can be obtained from the lymphocyte-rich fraction. In certain embodiments, the APC composition used for priming T cells is derived from the same subject as the T cells.

In various embodiments, the APCs prepared in accordance with a method disclosed herein can be used to prime and expand T cells directed to the one or more selected antigen ex vivo, the method comprising co-culturing of the APCs with the T cells.

In certain embodiments, a lymphocyte-rich fraction of PBMCs can be co-cultured with the tolerogenic APCs disclosed herein (e.g., at a ratio between about 1:1 to about 40:1) to expand Treg cells that are reactive to the one or more epitopes. Treg cells directed to the one or more epitopes presented on the APCs preferentially expand and enrich during the co-culturing process, resulting in a primed and expanded T cell composition directed to the one or more epitopes. In certain embodiments, the T cell composition may be re-stimulated by the same population of antigen-pulsed APCs. In certain embodiments, the T cell composition may be re-stimulated by another population of APCs pulsed by the same antigens or peptides (e.g., a fresh population of APCs produced by the same method as those used in initial stimulation).

In certain embodiments, the primed and expanded Treg cells are CD8+CD122^{hi} CD49d/f+, CD8+CD28−, CD8+CD103+CD25^{hi}, CD8+FOXP3+, CD8+FOXP3+Helios+, or CD8+CD45RC^{low}. The resulting expanded and primed T cells can be used for various purposes (e.g., in T cell therapies as further disclosed herein).

### V. Pharmaceutical Compositions and Therapeutic Methods

Disclosed herein are pharmaceutical compositions that contain one or more peptides, nucleic acids, APCs, T cells, or fusion proteins described herein that are useful for treating T1D. Exemplary pharmaceutical compositions described herein include but are not limited to peptide compositions, nucleic acid compositions, presentation agents (e.g., APCs and artificial APCs), and T cell compositions. Also disclosed are therapeutic uses of these pharmaceutical compositions.

### Tolerogenic Peptide or Nucleic Acid Compositions

The T1D-associated peptides disclosed herein are useful as tolerogenic compositions for treating and preventing T1D in a subject.

Accordingly, in one aspect, the present disclosure provides a pharmaceutical composition (e.g., tolerogenic peptide composition or nucleic acid composition) comprising a peptide composition disclosed herein (see the “Peptide Composition” subsection in Section II above) or nucleic acids encoding the peptide. Typically, the peptide(s) are delivered to a target cell. In some aspects, a nucleic acid encoding the peptide(s) in a nucleic acid composition are delivered to a target cell and encoded to the peptide(s). Accordingly, in certain embodiments, the pharmaceutical composition further comprises a vehicle, e.g., a delivery agent, for delivering the T1D-associated peptide(s) or nucleic acids encoding the peptide(s) to a target cell (e.g., tolerogenic target cell). It is understood that the target cell, after internalizing the peptide(s), can present the peptide(s) on the cell surface with a cognate MHC (e.g., HLA-A*02:01), thereby to stimulate tolerogenic T cell responses. Delivery agents for delivering peptides or nucleic acids to target cell (e.g., tolerogenic target cell) include but are not limited to nanoparticles, liposomes, virus-like particles, and red blood cells. The delivery agent may also reduce susceptibility of the peptides or nucleic acids to proteolytic degradation.

In certain embodiments, the delivery agent comprises a nanoparticle. In certain embodiments, the nanoparticle is a polymeric nanoparticle composed of poly(lactide-co-glycolide) (PLG or PLGA) (Jamison et al., J. Immunol. (2019) 203(1) 48:57), polylactic acid (PLA) (Ruan et al., J. Control Release (2018) 279:306-315), or poly(ε-caprolactone) (PCL) (Kim et al., Polymers (Basel) (2019) 11(2): 288). In certain embodiments, the polymeric nanoparticle is an amphiphilic block copolymer where at least one block is hydrophobic and one block is hydrophilic and the block copolymer self-assembles in an aqueous solution. The amphiphilic block copolymer can be a di-block A-B, tri-block A-B-A or a graft copolymer and can form polymeric micelles. The hydrophobic portion of the block copolymer forms the core of micelles, while the hydrophilic portion forms the shell or the corona. The commonly used hydrophilic blocks are poly(ethylene glycol), poly(N-vinyl pyrrolidine) (PVP), polysialic acid (PSA) (Lei et al., Chem Sci. (2021) 12(18): 6449-6457), and poly(N-isopropyl acrylamide) (pNIPAAm) among others. Hydrophobic blocks can comprise poly(propylene oxide) (PPO), PCL, PLGA, poly(L-lactide), poly(L-lysine) (Luo et al., J. Control Release (2013) 170(2): 259-67) poly(L-aspartic acid) (pAsp) (Kagaya et al., Gene Ther (2012) 19(1): 61-9), and poly(L-histidine) (pHis) (Wu et al., Biomaterials (2013) 34(4): 1213-22). In some embodiments the nanoparticle is a phospholipid micelle, where the hydrophobic block of polymeric micelles can be short, phospholipid residues like disteroyl phosphatidyl ethanolamine (DSPE) conjugated chemically to hydrophilic blocks like mPEG to form mPEG-DSPE micelles (Banerjee et al., Nanomedicine (2013) 9(6): 722-8; Kumar et al., ACS Infect Dis. (2019) 5(3): 443-453). Polymeric nanoparticles can be formed by the double emulsion or nanoprecipitation methods. In certain embodiments, the nanoparticle is a gold particle with a layer of thiol-poly-ethylene glycol (PEG) (Yeste et al., Sci Signal. (2016) 9(433): ra61). In certain embodiments, the gold nanoparticle is 60 nm in diameter. In certain embodiments, the nanoparticle is a liposome or a nanoliposome prepared by the thin-film hydration or ethanol injection method (Kenison et al., Proc. Natl. Acad. Sci. (2020) 117(50): 32017-32028, WO2019/165436 A1). In certain embodiments, the nanoparticle comprises a polystyrene particle or a polystyrene microsphere. Peptides or nucleic acids can be encapsulated within the nanoparticles either by passive loading through spontaneous self-assembly (Luo et al., J Control Release. (2013) 10:170(2): 259-67, Kenison et al., Proc. Natl. Acad. Sci. (2020) 117(50): 32017-32028, Pujol-Autonell et al., PLOS One (2015) 10(6): e0127057), electrostatically attached to the nanocarrier through interaction of opposite charges on the antigen and nanoparticle, physically adsorbed to nanoparticles (US2018/0071376 A1) or can be covalently attached to nanoparticle surface using well-defined conjugation chemistries as described in Bioconjugate Techniques, 3rd edition by Greg T Hermanson, Academic Press Inc. 2013) (US 2019/0282707 A1). The nanoparticles may incorporate functional groups for further reaction. Functional groups include electrophiles or nucleophiles which can conveniently react with other molecules. Examples of nucleophiles are primary amines, thiols, and hydroxyl groups. Examples of electrophiles are succinimidyl esters, aldehydes, isocyanates, and maleimides (US2019/0282707A1, US 2020/0046816A1). Functional groups can be readily incorporated in the PEG moiety that is used to decorate or graft the surface of nanoparticles and antigens.

In certain embodiments, the nanoparticle has a diameter that makes the particle suitable for systemic, in particular parenteral, administration of nucleic acids or peptides, typically a diameter less than 1,000 nanometers (nm). In some embodiments, the nanoparticle has a diameter of less than 600 nm. In some embodiments, the nanoparticle has a diameter of less than 400 nm. In some embodiments, the nanoparticle has a diameter in the range of 50 to 1,000 nm, for example, 50 to 900 nm, 50 to 800 nm, 50 to 700 nm, 50 to 600 nm, 50 to 500 nm, 50 to 400 nm, 50 to 300 nm, 50 to 200 nm, 50 to 100 nm, 100 to 1,000 nm, 100 to 900 nm, 100 to 800 nm, 100 to 700 nm, 100 to 600 nm, 100 to 500 nm, 100 to 400 nm, 100 to 300 nm, 100 to 200 nm, 150 to 1,000 nm, 150 to 900 nm, 150 to 800 nm, 150 to 700 nm, 150 to 600 nm, 150 to 500 nm, 150 to 400 nm, 150 to 300 nm, 150 to 200 nm, 200 to 1,000 nm, 200 to 900 nm, 200 to 800 nm, 200 to 700 nm, 200 to 600 nm, 200 to 500 nm, 200 to 400 nm, 200 to 300 nm. In some embodiments, the nanoparticle has a diameter in the range of 250 to 550 nm, 300 to 500 nm, or 200 to 400 nm.

Nanoparticles can be targeted to certain cells (e.g., tolerogenic cells) by various means. Tolerogenic APCs can be found in the liver and spleen. Accordingly, in certain embodiments, the nanoparticle preferentially targets the liver. In certain embodiments, the nanoparticle preferentially targets the spleen, for example, by having a negative zeta potential (see WO 2013/192532, US 2019/0282707 A1, Kranz et al., Nature (2016) 534(7607): 396-401). In certain embodiments, the nanoparticle comprises an apoptotic body surrogate (see WO2013/184976). In certain embodiments, the nanoparticle comprises one or more apoptotic signaling molecules selected from annexin-1, annexin-5, milk fat globule-EGF-factor 8 (MFG-E8), calreticulin, phosphatidylserine, CD47, oxidized LDL, Fas-ligand and TNF. In certain embodiments, the nanoparticle can be targeted to immune cells based on its particle size (Thorp et al., Front Immunol. (2020) 11:945, US 2019/0282707 A1). In certain embodiments the nanoparticle has a particle size of at least 200 nm, which allows particles to accumulate predominantly in the red pulp of the spleen and preferentially interact with immune cells therein. A particle size of 200 nm has been shown to preferentially accumulate in late endosomal or lysosomal compartments, making them ideal for immunomodulatory properties, as cargo movement through the late endosomal compartment via intracellular endosomal receptors is an important step for engaging adaptive and innate immune processes (Rejman et al., Biochem J. (2004) 377 (Pt 1): 159-69, Gleeson., Semin Cell Dev Biol. (2014) 31:64-72). In certain embodiments, the particle has a diameter in the range of 50-800 nm, for example, 50-700 nm, 50-600 nm, 50-500 nm, 50-400 nm, 50-300 nm, 50-200 nm, 50-150 nm, 50-100 nm, 50-90 nm, 50-80 nm, 50-70 nm, 50-60 nm, 100-800 nm, 100-700 nm, 100-600 nm, 100-500 nm, 100-400 nm, 100-300 nm, 100-200 nm, 100-150 nm, 150-800 nm, 150-700 nm, 150-600 nm, 150-500 nm, 150-400 nm, 150-300 nm, 150-250 nm, 150-200 nm, 200-800 nm, 200-700 nm, 200-600 nm, 200-500 nm, 200-400 nm, 200-300 nm.

In certain embodiments, the nanoparticle comprises a ligand (e.g., an antibody or an antigen-binding fragment thereof) that binds an antigen of a target cell (e.g., tolerogenic cell). In certain embodiments, the ligand comprises an antibody that binds such an antigen or an antigen-binding fragment thereof.

In certain embodiments, the delivery agent comprises a liposome. See, e.g., U.S. Pat. Nos. 4,235,871; 5,000,887; 5,395,619; 4,837,028; U.S. Patent Applications Nos: US 2016/0324779A1, US 2020/0405642A1, and EP Patent No: EP3,095,440. Liposomes are lipid bilayer spherical membranes that provide both hydrophilic and hydrophobic environments. In certain embodiments, the liposome is a multilamellar liposomes/vesicles (MLV), an oligolamellar vesicles (OLV), a multilamellar liposomes/vesicles (MVV), or an unilamellar vesicles (ULV). In certain embodiments, the liposome comprises neutral lipids or negatively charged lipids. In certain embodiments, the liposome comprises dilaurylphosphotidylcholine (DLPC), dimyristoyl phosphotidylcholine (DMPC), dipalmitoylphosphotidylcholine (DPPC), dioleolylphosphotidyl choline (DOPC), dilaurylphosphotidylethanolamine (DLPE), dipalmitoylphosphotidylcholine (DPPC), distearoylphosphotidylcholine (DSPC), dioleolylphosphotidylcholine (DOPC), dimyristoyl phosphotidylethanolamine (DMPE), distearoylphosphotidylethanolamine (DSPE), dilaurylphosphotidylglycerol (DLPG), dicetylphosphate (DCP), dioleoylphosphatidylethanolamine (DOPE), 1,2-dioleoyl-3 trimethylammoniumpropane (DOTAP), dioleoylphosphatidylserine (DOPS), hydrogenated soybean phosphatidylcholine (HSPC), or cholesterol In certain embodiments, the liposome comprises PEG lipids. In certain embodiments, the liposome comprises L-α-egg phosphatidylcholine (EPC) and/or L-α-egg phosphatidylglycerol (EPG). In some embodiments, the liposomes comprise 1,2-dioleoyl-sn-glycero3-phospho-L-serine (DOPS), 1,2-didodecanoyl-sn-glycero-3-phosphocholine (DLPC), and cholesterol. In some embodiments, the liposomes are negatively charged.

In some embodiments, the liposomes are nanoliposomes. In some embodiments, the diameter of the liposome is 50-500 nm, for example, 50-400 nm, 50-300 nm, 50-200 nm, 50-100 nm, 100-500 nm, 100-400 nm, 100-300 nm, 100-200 nm, 200-500 nm, 200-400 nm, 200-300 nm, 300-500 nm, 300-400 nm, or 400-500 nm.

Liposomes can be targeted to target cells (e.g., tolerogenic cells) by various means. In certain embodiments, the liposome comprises phosphatidylserine. In certain embodiments, the liposome comprises a ligand (e.g., an antibody an antigen-binding fragment thereof) that binds an antigen of a target cell (e.g., tolerogenic cell), as disclosed above in connection with nanoparticles.

In certain embodiments, the delivery agent comprises a targeting domain (e.g., a natural ligand, an antibody or an antigen-binding fragment thereof) that binds a receptor that is specifically or preferentially expressed on tolerogenic APCs, relative to immunogenic APCs. Exemplary receptors include but are not limited to MARCO, ILT-3, ILT-4, and CD52. Additional receptors comprise Clec9A, an anti-C-type lectin (e.g., DC-SIGN, Mannose receptor (MR), or macrophage galactose-type C-type lectin (MGL)), dendritic cell immunoreceptor (DCIR), DCIR2, DEC205 (CD205), Langerin (CD207), and Sialic-acid binding immunoglobulin-type lectin (Siglec).

In certain embodiments, the delivery agent comprises a erythroid cell such as a red blood cell. Erythroid cells can be engineered to include a wide variety of exogenous therapeutic proteins (see, e.g., WO 2019/133881). In certain embodiments, the erythroid cell is an enucleated mature red blood cell that lacks a nucleus. In certain embodiments, the erythroid cell is CD47-negative.

Tolerance to ingested antigens is an essential feature required for normal homeostasis and maintenance of barrier function in the gastrointestinal tract. Therefore, oral tolerance is another means of suppressing T cell immunity. In certain embodiments, the delivery agent comprises a carrier for delivering a peptide or nucleic acid to the small intestine, for example, a coating that renders resistance to degradation in the low pH and the presence of proteolytic enzymes in the stomach. Also contemplated herein is a peptide or nucleic acid composition suitable for oral administration without a delivery agent, for example, if the peptide is designed and/or chemically modified to resist degradation in the stomach.

In certain embodiments, the pharmaceutical composition (e.g., tolerogenic peptide composition, tolerogenic nucleic acid composition, nanoparticle composition, liposome composition, etc.) further comprises an immunomodulator. An immunomodulator can be an immunosuppressor, which reduces effector cell (e.g., cytotoxic T cell) activity, or an immunostimulator, which promotes T cell anergy or exhaustion (e.g., described in Kwong et al., Immunology & Cell Biology (2021) 99:486-495, and Linsley et al., Curr Opin Endocrinol Diabetes Obes (2019) 26:213-218) It is contemplated that the immunomodulator, when delivered to or expressed by the target cell, may induce or stabilize a tolerogenic, anergic, or exhausted status of the target cell or a cell in the vicinity. In certain embodiments, the immunomodulator comprises an immunomodulatory cytokine, for example, an immunomodulatory cytokine selected from IL-2, IL-10, TGF-β, IL-37, IL-27, IL-35, IL-31, Vasoactive Intestinal Peptide (VIP), and variants thereof. In one embodiment, the immunomodulatory cytokine is a mutant IL-2 that preferentially activates the IL-2Rαβγ receptor complex relative to the IL-2Rβγ receptor complex (see, e.g., Ghelani et al., Front. Immunol. (2020) 11:1106; Khoryati et al., Sci Immunol. (2020) 5(50): eaba5264). In certain embodiments, the immunomodulator comprises a nucleic acid encoding an immunomodulatory cytokine. In certain embodiments, the immunomodulator comprises a nucleic acid encoding an intracellular or transmembrane immunomodulatory protein, for example, an immunomodulatory protein selected from PD-L1, PD-L2, ICOS ligand, ILT3, ILT4, BTLA, Fas, CD39, and indoleamine 2,3-dioxygenase 1 (IDO1), heme oxygenase 1, HLA-G, CD95L, galectin-1, and DC-SIGN. In certain embodiments, the immunomodulator comprises an immunomodulatory compound, for example, an immunomodulatory compound selected from vitamin A, vitamin D (e.g., 1α,25-dihydroxyvitamin D3, a.k.a. calcitriol), adenosine, kynurenine, retinoic acid, rapamycin, dexamethasone, corticosteroids, and a AhR ligand such as 2-(1′ H-indole-3′-carbonyl)-thiazole-4-carboxylic acid methyl ester (ITE). In certain embodiments, the immunomodulator provides a tolerogenic signal to an APC (e.g., dendritic cell, macrophage). In certain embodiments, the immunomodulator comprises a ligand (for example a carbohydrate ligand) or an antibody to APC receptors, for example, Clec9A, an anti-C-type lectin (e.g., DC-SIGN, Mannose receptor (MR), or macrophage galactose-type C-type lectin (MGL)), dendritic cell immunoreceptor (DCIR), DCIR2, DEC205 (CD205), Langerin (CD207), and Sialic-acid binding immunoglobulin-type lectin (Siglec). The immunomodulator can be delivered to the target cell by the delivery agent, for example, where the immunomodulator is present in or on the delivery agent. Depending on the form of the immunomodulator and delivery agent, the immunomodulator can be encapsulated or otherwise contained in the delivery agent, or covalently or non-covalently attached to an outer or inner surface of the delivery agent. In certain embodiments, the immunomodulator is disposed in the delivery agent.

In certain embodiments, the tolerogenic composition further comprises a pharmaceutically acceptable carrier, excipient, or stabilizer (see, e.g., Adeboye Adejare, Remington: The Science and Practice of Pharmacy (23d ed. 2020)). Suitable carriers are well known in the art and may include for example, nanoparticles, nanotubes, dendrimers, liposomes, foams, hydrogels, cubosomes, quantum dots, natural drug carriers, exosomes, and macrophages. Acceptable carriers, excipients, or stabilizers are nontoxic to the recipients at the dosages and concentrations, and may comprise phosphate buffered saline solutions, water, emulsions, such as oil/water emulsions, various types of wetting agents, and sterile solutions. In some embodiments, the carrier or excipient comprises buffers such as phosphate, citrate, and other organic acids; antioxidants including ascorbic acid and methionine; preservatives (such as Mercury((o-carboxyphenyl)thio)ethyl sodium salt (THIO E SAL), octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG), TWEEN or PLURONICS.

Peptide compositions comprising such carrier, excipients, or stabilizers can be formulated by well-known conventional methods. In some embodiments, the tolerogenic peptide composition is formulated to have a pH in the range of about 4 S to about 9.0, including for example pH ranges of about any one of 5.0 to about 8.0, about 6.5 to about 7.5, or about 6.5 to about 7.0. In some embodiments, the tolerogenic peptide composition can also be made to be isotonic with blood by the addition of a suitable tonicity modifier, such as glycerol. In some embodiments, the tolerogenic peptide composition is formulated as a liquid or the liquid is dried for a solid preparation (for example by spray drying or lyophilization). The lyophilization process is aimed at removal of water and typically involves freezing, primary drying, and secondary drying. To safeguard the peptide molecules from such stresses, typically cryoprotectants are used to improve viability and structural stability. Cryoprotectants include, but are not limited to DMSO, sugars like trehalose and sucrose, polysaccharides, like starch and dextran, or proteins. Solid preparations allow for a long storage time of the tolerogenic peptide composition. Lyophilized preparations of the tolerogenic peptide composition can be stored in bulk or in ready-made doses for use such as in a vial. The content of peptide for a one dose vial may be 1, 2, 3, 4, 5, 6, 7, 8, 9, 10, 11, 12, 13, 14, 15, 16, 17, 18, 19, or 20 mg. The content of peptide for a one dose vial can be 1.0 to 20 mg, 2.0 to 15 mg, 5.0 to 14 mg, 10 to 13 mg, or 12 mg. Before administration to a patient, the solid preparation can be dissolved with a suitable solvent such as water.

These pharmaceutical compositions can be administered to the subject at a suitable dose. It is preferred that the pharmaceutically acceptable carrier be one which has no detrimental side effects or toxicity under the conditions of use. Preferably, a tolerogenic peptide composition is sterile and produced according to GMP guidelines.

In some embodiments, the pharmaceutical composition, e.g., a tolerogenic peptide or nucleic acid composition, is in the form of a lyophilized formulation or an aqueous solution. The composition can be in dosages suspended in any appropriate pharmaceutical carrier in sufficient volume to carry the dosage. Generally, the final volume, including carriers, adjuvants, and the like, typically will be at least 0.5 mL. The upper limit is governed by the practicality of the amount to be administered, generally in the range of about 0.5 ml to about 2.0 ml. In some embodiments, the pharmaceutical composition, e.g., a tolerogenic peptide or nucleic acid composition, is administered to a patient enterally or parenterally. Depending upon the circumstances, the tolerogenic peptide or nucleic acid composition can be administered to a patient by oral, sublingual, gastric, or rectal administration. Alternatively, the tolerogenic peptide or nucleic acid composition can administered to a patient by intravenous, intramuscular, intratumoral, intradermal, intrajejunal, intraileal, intracolonic, or intrarectal administration. The tolerogenic peptide or nucleic acid composition can be also delivered by subcutaneous axillary and/or inguinal injection.

Nucleic acids encoding for the peptides and T cell epitopes disclosed herein can also be useful for treating and preventing T1D in a subject. Accordingly, in another aspect, the present disclosure provides a pharmaceutical composition (e.g., tolerogenic composition) comprising one or more nucleic acids encoding one or more peptides of a peptide composition disclosed herein (see the “Peptide Compositions” subsection of Section II above). Where the peptides or polypeptides constitute a fragment of the complete antigen protein, such tolerogenic nucleic acid compositions are also known as minigene vaccines.

In some embodiments, at least one nucleic acid of the nucleic acid composition encodes a plurality of different peptides. The nucleic acid can be engineered to insert protease cleavage sites and/or ribosomal skipping elements between the peptides, such that the encoded peptides are processed to produce separate peptides. Such polynucleotide constructs, also called “strings,” are useful as tolerogenic nucleic acid compositions (see, e.g., Velders et al., J. Immunol. (2001) 166:5366-73; Schubert et. al., Genome Medicine (2016) 8:9). In some embodiments, the composition comprises one or more polynucleotide constructs that encode one or more of the peptides disclosed herein with one or more protease cleavage sites and/or ribosomal skipping elements between the peptides. Both coding and non-coding strands can be incorporated into the composition. In some embodiments, the string refers to a polynucleotide chain that encodes a plurality of the peptides in tandem. In some embodiments, the string encodes 2 to 100, 2 to 90, 2 to 80, 2 to 70, 2 to 60, 2 to 50, 2 to 40, 2 to 30, 2 to 20, 2 to 10, 10 to 100, 10 to 90, 10 to 80, 10 to 70, 10 to 60, 10 to 50, 10 to 40, 10 to 30, 10 to 20, 20 to 100, 20 to 90, 20 to 80, 20 to 70, 20 to 60, 20 to 50, 20 to 40, 20 to 30, 50 to 100, 50 to 90, 50 to 80, 50 to 70, or 50 to 60 peptides. In some embodiments, the peptides are arranged on a string to maximize recognition by HLA-A*02:01. In some embodiments the epitope-coding sequences in a string construct are flanked by one or more sequences selected for better cleavability for peptide presentation by MHCs, better expression, and/or improved translation in a cell in a subject. The flanking sequences may comprise ribosomal skipping elements such as a T2A, P2A, F2A, or E2A sequence. In some embodiments, the one or more cleavage sequences are selected from the group consisting of FRAC, KRCF, KKRY, ARMA, RRSG, MRAC, KMCG, ARCA, KKQG, YRSY, SFMN, FKAA, KRNG, YNSF, KKNG, RRRG, KRYS, and ARYA. In some embodiments, the amino acid sequences encoded by a string construct further comprises a signal protein sequence. In some embodiments the string constructs may be mRNA. In some embodiments, a pharmaceutical composition may comprise one or more mRNA string constructs.

In some embodiments, the tolerogenic nucleic acid composition is a DNA composition (e.g., tolerogenic DNA composition). The DNA composition may comprise a DNA plasmid or a minicircle DNA encoding for one or more antigen peptides and/or T cell epitopes disclosed herein. The DNA plasmid can further comprise a promoter sequence and a backbone sequence. The DNA composition can be formulated with nano-carriers to shield the DNA composition from degradation by DNases and other enzymes. Suitable nano-carriers for DNA compositions are well known in the art and may include for example, nanoparticles, nanotubes, dendrimers, liposomes, foams, hydrogels, cubosomes, quantum dots, natural drug carriers, and exosomes.

Depending upon the circumstances, the DNA composition is administered to a patient by intravenous injection, oral, or pulmonary administration. Alternatively, the DNA composition can be applied topically via the skin or intramuscularly. Alternatively, the DNA composition can be administered by intramuscular (IM) injection, subcutaneous injection, or intradermal injection. Regardless of the injection site, the pDNA transfects cells in the subject, which can then undergo a type of programmed cell death known as apoptosis. A cell that undergoes apoptosis releases small membrane-bound fragments that known as apoptotic bodies, which trigger the endocytosis of cellular debris by immature dendritic cells (iDC). The activity of iDCs can then initiate the generation of exogenous antigens, which are exclusively presented by class II MHCs. In addition to acting on either myocytes or keratinocytes, an administration route can also transfect APCs located near the injection site. This direct transfection route results in endogenous transgene expression and presentation of the antigen peptide through both class I and class II MHCs, thereby yielding both CD8+ and CD4+ T cells.

In some embodiments, the tolerogenic nucleic acid composition is an RNA composition (e.g., tolerogenic RNA composition) encoding for one or more antigen peptides and/or T cell epitopes disclosed herein. In some embodiments the RNA composition is a messenger RNA (mRNA) composition. The RNA composition optionally further comprises one or more chemical modifications. For example, the RNA composition can comprise a five-prime (5′) and a three-prime (3′) untranslated region (UTR), and can be further stabilized by 7-methylguanosine (m7G) 5′ cap and 3′ poly(A) tails respectively, and/or a suitable Poly(A) sequence. In some embodiments, the mRNA composition comprises modified nucleosides, for example Pseudouridine (Ψ), 1-methylpseudouridine (m1Ψ), and 5-methylcytidine (m5C). In some embodiments, the RNA composition comprises a self-amplifying RNA (saRNA), which further encodes an RNA-dependent RNA polymerase that can be translated in situ, thereby amplifying the peptide- or antigen-encoding sequence(s) in situ (see, e.g., Bloom et al., Gene Therapy (2021) 28:117-29). In some embodiments, the mRNA composition can be formulated into delivery agents, including but not limited to lipid nanoparticles, polymers, liposomes, and peptides. An overview of mRNA compositions such as mRNA cancer vaccines is provided, for example, in Miao, L. et al., Molecular Cancer (2021) 20:41.

In certain embodiments, the tolerogenic composition comprises both an antigen peptide and a nucleic acid coding for the peptide. In some embodiments, the antigen peptide and the nucleic acid are linked.

In certain embodiments, the pharmaceutical composition further comprises a delivery agent for delivering the nucleic acid(s) to a target cell (e.g., tolerogenic target cell). It is understood that the target cell, after internalizing the peptide(s), can present the peptide(s) on the cell surface with a cognate MHC (e.g., HLA-A*02:01), thereby to stimulate tolerogenic T cell responses. Delivery agents for delivering nucleic acids to target cell (e.g., tolerogenic target cell) include but are not limited to lipid nanoparticles (“LNPs”).

In certain embodiments, the delivery agent comprises a LNP. In some embodiments, the lipid comprises a cationic lipid, e.g., a ionizable cationic lipid. In one embodiment, the lipid forms a complex with and/or encapsulates the nucleic acid (e.g., RNA). Nanoparticulate RNA formulations with defined particle size in which the net charge of the particles is close to zero or negative, such as electroneutral or negatively charged lipoplexes of RNA and liposomes, for example, lipoplexes comprising DOTMA and DOPE or DOTMA and DOTMA and Cholesterol, can lead to substantial supply of RNA to the spleen's DCs after systemic administration. In certain embodiments, in a nanoparticulate RNA formulation, the ratio of positive charges to negative charges on the nanoparticles is 1.4:1 or less, and/or the zeta potential of the nanoparticles is 0 or less. In certain embodiments, the ratio of positive charges to negative charges in the nanoparticles is between 1.4:1 and 1:8, for example, between 1.2:1 and 1:4, between 1:1 and 1:3, between 1:1.2 and 1:2, between 1:1.2 and 1:1.8, between 1:1.3 and 1:1.7, between 1:1.4 and 1:1.6, or about 1:1.5. In certain embodiments, the zeta potential of the nanoparticles is −5 mV or less, −10 mV or less, −15 mV or less, −20 mV or less, or −25 mV or less. In certain embodiments, the zeta potential of the nanoparticle is −35 mV or greater, −30 mV or greater, or −25 mV or greater. In certain embodiments, the nanoparticles have a zeta potential of −50 to 0 mV, for example, −40 to 0 mV, −30 to −10 mV. In certain embodiments, positive charges are contributed by at least one cationic lipid present in the nanoparticles; negative charges are contributed by RNA and certain lipid components of the LNPs such as anionic helper lipids. In certain embodiments, the nanoparticles comprise at least one helper lipid, such as a neutral lipid or an anionic lipid.

In certain embodiments, the nanoparticles are lipoplexes that comprise DOTMA and DOPE in a molar ratio of 10:0 to 1:9, for example, 8:2 to 3:7, or 7:3 to 5:5, and where the ratio of charges from positive charges in DOTMA to negative charges in RNA is 1.8:2 to 0.8:2, for example, 1.6:2 to 1:2, 1.4:2 to 1.1:2, or about 1.2:2. In certain embodiments, the nanoparticles are lipoplexes that comprise DOTMA and cholesterol in a molar ratio of 10:0 to 1:9, for example, 8:2 to 3:7 or 7:3 to 5:5, and where the ratio of charges from positive charges in DOTMA to negative charges in RNA is 1.8:2 to 0.8:2, for example, 1.6:2 to 1:2, 1.4:2 to 1.1:2, or approximately 1.2:2. In certain embodiments, the nanoparticles are lipoplexes that comprise DOTAP and DOPE in a molar ratio of 10:0 to 1:9, for example, 8:2 to 3:7 or 7:3 to 5:5, and where the ratio of charges from positive charges in DOTMA to negative charges in RNA is 1.8:2 to 0.8:2, for example, 1.6:2 to 1:2, 1.4:2 to 1.1:2, or approximately 1.2:2. In certain embodiments, the nanoparticles are lipoplexes that comprise DOTMA and DOPE in a molar ratio of 2:1 to 1:2, for example, 2:1 to 1:1, and in which the ratio of positive charges in DOTMA to negative charges in RNA is 1.4:1 or less. In certain embodiments, nanoparticles are lipoplexes that comprise DOTMA and cholesterol in a molar ratio of 2:1 to 1:2, for example, 2:1 to 1:1, and in which the ratio of positive charges in DOTMA to negative charges in RNA is 1.4:1 or less. In certain embodiments, nanoparticles are lipoplexes that comprise DOTAP and DOPE in a molar ratio of 2:1 to 1:2, for example, 2:1 to 1:1, and in which the ratio of positive charges in DOTAP to negative charges in RNA is 1.4:1 or less.

In certain embodiments, the composition (e.g., LNP composition) further comprises an immunomodulator, such as an immunomodulator described in the “Tolerogenic Peptide Compositions” subsection above.

In some embodiments, the tolerogenic nucleic acid composition further comprises one or more carriers or excipients. Exemplary excipients are described herein (see the “Tolerogenic Peptide Compositions” subsection above).

Nucleic acid compositions comprising such adjuvants, carriers, and/or excipients can be formulated by well-known conventional methods. These pharmaceutical compositions can be administered to the subject at a suitable dose. It is preferred that the pharmaceutically acceptable carrier be one which has no detrimental side effects or toxicity under the conditions of use. Preferably, a tolerogenic nucleic acid composition is sterile and produced according to GMP guidelines.

In some embodiments, the tolerogenic nucleic acid composition is in the form of a lyophilized formulation or an aqueous solution. The tolerogenic nucleic acid composition can be in dosages suspended in any appropriate pharmaceutical carrier in sufficient volume to carry the dosage. Generally, the final volume, including carriers, adjuvants, and the like, typically will be at least 0.5 mL. The upper limit is governed by the practicality of the amount to be administered, generally in the range of 0.5 mL to about 4.0 mL, such as 0.5 mL to about 2.0 mL. In some embodiments, the tolerogenic nucleic acid composition is administered to a patient enterally or parenterally. In some embodiments, the tolerogenic nucleic acid compositions are administered to a patient by intravenous, intramuscular, intratumoral, intradermal, intrajejunal, intraileal, intracolonic, or intrarectal administration.

Suitable doses for tolerogenic nucleic acid compositions depend on the specific nucleic acid composition (such as DNA composition or RNA composition, or formulation in a lipid particle). Dosages for DNA compositions can be in the range of 0.1-50 mg, 0.1-20 mg, 0.1-10 mg, 0.5-5 mg, 0.5-2 mg, 0.5-1 mg, 1-50 mg, 1-20 mg, 1-10 mg, 1-5 mg, 1-2 mg, 2-20 mg, 2-10 mg, 2-5 mg, 5-50 mg, 5-20 mg, 5-10 mg, 10-50 mg, 10-20 mg, or 20-50 mg, for example, 0.5 mg, 1 mg, 1.5 mg, 2 mg, 2.5 mg, 3 mg, 3.5 mg, or 4 mg per dose. Dosages for RNA compositions can be in the range of 1-500 μg, 1-200 μg, 1-100 μg, 1-50 ug, 1-25 μg, 10-500 μg, 10-200 μg, 10-100 μg, 10-50 ug, 10-25 μg, 25-500 μg, 25-200 μg, 25-100 μg, 25-50 ug, 50-500 μg, 50-200 μg, 50-100 μg, 100-500 μg, 100-200 μg, or 200-500 μg, for example, 5 μg, 10 μg, 25 μg, 50 ug, 75 μg, 100 μg, 125 μg, 150 μg, or 200 μg per dose. Depending upon the circumstances, the tolerogenic nucleic acid composition can be administered once or administered weekly for at least 2, 3, or 4 weeks. In some embodiments, the tolerogenic nucleic acid composition is administered weekly for 4 weeks followed by monthly administrations for at least 2, 3, 4, 5, or 6 more months.

### Presentation Agents

The T1D-associated peptides disclosed herein can be presented by cognate MHCs (e.g., HLA-A*02:01) to form peptide-MHC (pMHC) complexes. The pMHC complexes bind T cells that are prevalent in T1D patients, and are thus useful for targeting T cells (e.g., CD8″ cytotoxic T cells) for treating and preventing T1D in a subject. Given that pMHC complexes generally have low binding affinities to cognate T cell receptors (TCRs), a vehicle, e.g., a presentation agent, can be used to multimerize or cluster pMHC complexes to increase their binding avidity. Accordingly, in one aspect, the present disclosure provides a composition (e.g., pharmaceutical composition) comprising a plurality of pMHC complexes disclosed herein bound to a presentation agent. Useful presentation agents, when bound to the pMHC complexes, can form, e.g., antigen-presenting cells (APCs), artificial APCs, or soluble complexes such as multimerized pMHC complexes.

In certain embodiments, the composition comprises an APC (e.g., dendritic cell, cultured cell line, artificial APC, nanoparticle, bead, liposome, or lipid bilayer scaffold) displaying T cell epitopes disclosed herein presented by cognate MHC (e.g., HLA-A*02:01) molecules on an outer surface of the APC. APCs are useful for eliciting a tolerogenic immune response, thereby to treat or prevent T1D in a subject. In certain embodiments, the epitope is presented by a soluble MHC, such as a soluble pMHC fusion protein disclosed herein (see the “Soluble MHC Complexes” subsection of Section II above).

It is contemplated that the APC (e.g., dendritic cell) can present one or more T cell epitopes disclosed herein (see the “Tolerogenic APCs” subsection above), which is useful as tolerogenic APC compositions. In one example, the APC is a cell genetically engineered to silence or knock out a gene that mediates an immunostimulatory or inflammatory response (e.g., allograft inflammatory factor-1 (AIF1), see Elizondo et al., J. Leukoc. Biol. (2019) 105(1): 123-30). Depending on the intended use, the tolerogenic APC composition can be formulated in a carrier solution and stored frozen in a cell cryopreservation medium. Cell cryopreservation media are known in the art, for example buffer solutions with varying amounts of DMSO such as CryoStor® CS5 or CryoStor® CS10. The tolerogenic APC composition can then be administered to the subject after thawing. It is contemplated that the APC can be an autologous cell, i.e., from a subject to be treated with the tolerogenic APC composition.

Examples of artificial APCs useful in the present disclosure are known in the art. In one example, the artificial APC comprises a cultured cell line presenting one or more T cell epitopes disclosed herein. The cell line can be engineered to express an MHC, e.g., HLA-A*02:01. In one specific example, the cell line comprises the K562 human erythromyeloid cell line or the murine NIH/3T3 fibroblast line. To activate and expand T cells comprising cognate TCRs to the T1D-associated peptides, T cells can be co-cultured with the engineered cell line for a certain period of time before harvesting. In addition, the cell line can be engineered to further express a molecule that provides signal 2, for example β7 family proteins, OX-40L, CD70, or 4-1BBL.

In another example, the artificial APC comprises a nanoparticle presenting one or more T cell epitopes disclosed herein. The nanoparticle can be coated with a pMHC or a soluble pMHC complex (see Perica et al., BBA Molecular Cell Research (2015) 1853(4): 781-90), e.g., a T1D-associated peptide presented by HLA-A*02:01. Forms of soluble pMHC complexes are described in the “Soluble pMHC Complexes” subsection above. The nanoparticle can comprise various materials, for example, a gold particle with a layer of thiol-poly-ethylene glycol (PEG), a irondextran nanoparticle, or an iron oxide nanoparticle (e.g., a superparamagnetic iron oxide nanoparticle) (see Tsai et al., Immunity (2010) 32(4): 568-80; Clemente-Casares et al., Nature (2016) 530(7591): 434-40; Umeshappa et al., Nat Commun. (2019) 10(1): 2150; Umeshappa et al., J Clin Invest. (2020) 130(4): 1823-29).

In another example, the artificial APC comprises a bead presenting one or more T cell epitopes disclosed herein. In certain embodiments, the nanoparticle is engineered to present an MHC, for example HLA-A*02:01. In certain embodiments, the bead comprises sepharose or latex (polystyrene) beads or irondextran microparticles (see e.g., Perica et al., BBA Molecular Cell Research (2015) 1853(4): 781-790). In certain embodiments, the bead is a biodegradable bead, made for example from biodegradable polymers, such as poly(lactic acid) (PLA), poly (glycolic acid) (PGA), and their co-polymer, and poly (lactic-co-glycolic acid) (PLGA).

In another example, the artificial APC comprises a liposome or a lipid bilayer scaffold presenting one or more T cell epitopes disclosed herein. In certain embodiments, the liposome or a lipid bilayer scaffold is engineered to present an MHC, for example HLA-A*02:01. In some embodiments, the lipid bilayer scaffold is a APC-mimetic scaffold (APC-ms). In one embodiment, the lipid bilayer is supported by mesoporous silica micro-rods. In certain embodiments, the micro-rods are coated with a cytokine, a lipid bilayer, a MHC and a molecule that provides signal 2 (see e.g., Cheung et al., Nature Biotechnology (2018) 36:160-169). In certain embodiments, the liposome is an aryl hydrocarbon receptor-activating nanoliposome (AhR-NLP) (see e.g., Kenison et al., PNAS (2020) 117(50): 32017-32018 and Rothhammer et al., Nature Reviews Immunology (2019) 19:184-197).

Another example of an aAPC is described in Li et al., J Clin Invest (2021) 131(23): e141051, and comprises covalently tethered peptide-MHC modules and immunomodulators linked to an Fc domain scaffold. The resulting construct can be used in vitro or in vivo to modulate T cells, for example, by injecting into a patient to stimulate or modulate T cells in the patient.

The nanoparticle has a diameter that makes the particle suitable for administration, for example, systemic, in particular parenteral, administration. Nanoparticles typically have a diameter less than 1,000 nanometers (nm). In some embodiments, the nanoparticle has a diameter of less than 600 nm. In some embodiments, the nanoparticle has a diameter of less than 400 nm. In some embodiments, the nanoparticle has a diameter in the range of 50 to 1,000 nm, for example, 50 to 900 nm, 50 to 800 nm, 50 to 700 nm, 50 to 600 nm, 50 to 500 nm, 50 to 400 nm, 50 to 300 nm, 50 to 200 nm, 50 to 100 nm, 100 to 1,000 nm, 100 to 900 nm, 100 to 800 nm, 100 to 700 nm, 100 to 600 nm, 100 to 500 nm, 100 to 400 nm, 100 to 300 nm, 100 to 200 nm, 150 to 1,000 nm, 150 to 900 nm, 150 to 800 nm, 150 to 700 nm, 150 to 600 nm, 150 to 500 nm, 150 to 400 nm, 150 to 300 nm, 150 to 200 nm, 200 to 1,000 nm, 200 to 900 nm, 200 to 800 nm, 200 to 700 nm, 200 to 600 nm, 200 to 500 nm, 200 to 400 nm, 200 to 300 nm.

In certain embodiments, the composition further comprises an immunomodulator. It is contemplated that the pMHC complex bound to the presentation agent is targeted to T cells that express cognate TCRs. The immunomodulator, when delivered to an outer surface of the target T cell or internalized by the target T cell, can suppress survival, proliferation, activation, and/or memory formation of the target T cells. Immunomodulators can also be designed to modulate other immune cells in the microenvironment. In certain embodiments, the immunomodulator is conjugated to the pMHC complex or the presentation agent, either covalently or noncovalently. In a specific embodiment, the immunomodulator is coated on an outer surface of the presentation agent. When the composition comprises a cell based APC (e.g., a natural APC or a cellular artificial APC), the immunomodulator can also be expressed by the APC.

In certain embodiments, the immunomodulator comprises an immunomodulatory cytokine, for example IL-2, IL-10, TGF-β, IL-37, IL-27, IL-35, IL-31, and Vasoactive Intestinal Peptide (VIP), or variants and fragments thereof. In certain embodiments, the cytokine variant is a variant with altered binding affinity to its cognate receptor, for example the cytokine comprises IL-2, and the IL-2 comprises a variant with reduced or no binding affinity for CD25/IL-2Rx. In certain embodiments, the cytokine has at least 95%, 96%, 97%, 98%, 99%, or 100% identity to a wild type human cytokine. In certain embodiments, the cytokine comprises a cytokine fused with another protein, such as a cytokine antibody fusion or cytokine receptor fusion (for example IL-15/IL-15Ra fusion).

In certain embodiments, the immunomodulator comprises a protein providing a co-inhibitory signal 2 to T cells, for example PD-L1, PD-L2, ICOS ligand, CD80, CD86, ILT3, ILT4, Fas, CD39, and indoleamine 2,3-dioxygenase 1 (IDO1). In certain embodiments, the immunomodulator comprises an agonist of an immunomodulatory receptor of a T cell, for example, PD-1, 4-1BB, CTLA-4, BTLA, LAG-3, TIM-3, TIGIT, CD2, or CD3. In certain embodiments, the immunomodulator comprises the extracellular portion of a natural ligand of the immunomodulatory receptor of a T cell (e.g., PD-L1 or PD-L2) or a functional fragment thereof, or an agonistic antibody that binds the immunomodulatory receptor of a T cell (e.g., anti-PD-1 antibody, e.g., novolumab or pembrolizumab, or an anti-CTLA-4 antibody, e.g., ipilimumab) or an antigen-binding fragment thereof. In certain embodiments, the immunomodulator inhibits the CD80/CD86-CD28 signaling. For example, in specific embodiments, the immunomodulator comprises an extracellular fragment of CTLA-4 (for example abatacept or belatacept), an antagonistic ligand or antibody to CD80 or CD86, or an antigen-binding fragment thereof.

In certain embodiments, the immunomodulator provides a tolerogenic signal to an APC (e.g., dendritic cell, macrophage), which may be present in the microenvironment. In certain embodiments, the immunomodulator comprises a tolerogenic compound, for example vitamin D3, retinoic acid, rapamycin, dexamethasone, corticosteroids, ligands of the aryl hydrocarbon receptor (AhR), or specific cytokines (such as IL-10, TGFB). In certain embodiments, the immunomodulator comprises a ligand (for example a carbohydrate ligand) or an antibody to APC receptors, for example, Clec9A, an anti-C-type lectin (e.g., DC-SIGN, Mannose receptor (MR), or macrophage galactose-type C-type lectin (MGL)), dendritic cell immunoreceptor (DCIR), DCIR2, DEC205 (CD205), Langerin (CD207), and a Sialic-acid binding immunoglobulin-type lectin (Siglec) protein.

In some embodiments, the pMHC-presentation agent composition further comprises one or more carriers, excipients, and/or stabilizers. Exemplary adjuvants, carriers, and excipients are described herein (see the “Tolerogenic Peptide Compositions” subsection above). In some embodiments, the pMHC-presentation agent composition is administered to a patient by intravenous, intramuscular, intratumoral, or intradermal administration.

### T Cell Therapies

The T cell compositions disclosed herein are useful as T cell therapies (e.g., adoptive T cell therapy). Accordingly, in another aspect, the present disclosure provides a T cell therapy comprising one or more T cell compositions disclosed herein (see Section IV above). In certain embodiments, the T cell therapy is a Treg cell therapy.

In certain embodiments, the T cell therapy is autologous, i.e., T cells obtained from a patient, after in vitro culture, are administered to the same patient. In certain embodiments, the T cell therapy is allogeneic, optionally wherein the T cells are genetically engineered to inactivate a component of class I MHC (e.g., β2M).

In some aspects, the T cell compositions comprises T cells that express T cell mimetics disclosed herein.

The T cell therapy can further comprise one or more carriers, excipients, and/or stabilizers. Exemplary carriers, excipients, and stabilizers are described herein (see the “Tolerogenic Peptide Compositions” subsection above).

### Treatment of T1D

The present disclosure provides methods of treating T1D comprising administering a therapeutically effective amount of a pharmaceutical composition, tolerogenic composition, or APC, e.g., aAPC, disclosed herein to a subject in need thereof.

It is contemplated that the therapies disclosed herein are useful in treating symptomatic or pre-symptomatic T1D. T1D disease progression is characterized by several stages of the disease that are characterized by a gradual loss of β-cell function over time. Stages include pre-stage 1, stage 1, stage 2, and stage 3. It is appreciated, however, that not all stages are present in all patients. In pre-stage 1, exposure to driving factors creates the conditions for β-cell autoimmunity to emerge. In stage 1, β-cell autoantibodies are persistent, but normoglycemia remains and there are no clinical symptoms. Throughout stage 2, the number of β-cell autoantibodies may induce dysglycemia but still without any diabetes symptoms. In stage 3, β-cell autoantibodies are predominant and clear symptoms of diabetes have emerged. As the disease progresses, β-cell function falls below the threshold required to maintain glucose control creating a requirement for insulin replacement therapy. Genetic and environmental risk are both included in the disease etiology.

T1D can be diagnosed by various methods. For example, the glycated hemoglobin (A1C) test measures the percentage of blood sugar attached to the oxygen-carrying protein in red blood cells (hemoglobin). The higher the blood sugar levels, the more hemoglobin has sugar attached. An A1C level of 6.5 percent or higher on two separate tests indicates diabetes. Additionally, random blood and fasting blood sugar tests can be performed. A blood sample is taken at a random time and may be confirmed by repeat testing. Blood sugar values are expressed in milligrams per deciliter (mg/dL) or millimoles per liter (mmol/L). Regardless of the last meal, a random blood sugar level of 200 mg/dL (11.1 mmol/L) or higher suggests diabetes, especially when coupled with any of the signs and symptoms of diabetes, such as frequent urination and extreme thirst. For the fasting blood sugar test a blood sample will be taken after an overnight fast. A fasting blood sugar level less than 100 mg/dL (5.6 mmol/L) is normal. A fasting blood sugar level from 100 to 125 mg/dL (5.6 to 6.9 mmol/L) is considered prediabetes. If the blood sugar level is 126 mg/dL (7 mmol/L) or higher on two separate tests, suggests diabetes. Additional autoantibody tests (for example for Islet Cell Cytoplasmic Autoantibodies (ICA), Glutamic Acid Decarboxylase Autoantibodies (GADA), Insulinoma-Associated-2 Autoantibodies (IA-2A), Insulin Autoantibodies (IAA) and/or Zinc Transporter-8 Autoantibodies (ZnT8)) can confirm T1D.

In some aspects, the present disclosure provides a method of treating or preventing Type I Diabetes in a subject in need thereof comprising administering to the subject an effective amount of a means for inducing a tolerogenic immune response in combination with a pharmaceutically acceptable carrier. In some aspects, the tolerogenic immune response is against one or more T cell epitopes disclosed herein.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. In certain embodiments, delivery is such that the reduction of a symptom, or other parameter related to the disorder is greater than what would be observed with one treatment delivered in the absence of the other. The effect of the two treatments can be partially additive, wholly additive, or greater than additive.

The disclosure provides a method of treating T1D in a subject by the administration of a second therapeutic agent in combination with one or more of the therapies disclosed herein. Exemplary second therapeutic agents that may be used as part of a combination therapy, include, for example, subcutaneous injections of insulin combined with continuous glucose monitoring technology, insulin/glucagon delivery systems such as an artificial pancreas, metformine, pramlitide, glucagon-like peptide-1 receptor agonists (GLP-RAs) combined with insulin, sodium-glucose co-transporter inhibitors (SGLTi), tissue transplants, gene therapy, stem-cell therapies, and nutrition therapy.

The amount of the tolerogenic composition or artificial APC therapy disclosed herein and the amount of the additional therapeutic agent, and the relative timing of administration, may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, simultaneously, or together. Further, for example, the tolerogenic composition or artificial APC therapy may be administered during a time when the additional therapeutic agent(s) exerts its prophylactic or therapeutic effect, or vice versa.

### VI. Diagnostic Methods

Information about the immunoprevalent T cell epitopes associated with T1D can be used to diagnose or predict T1D in a subject. It has been discovered that CD8⁺ T cells that bind these epitopes are present at high frequencies across T1D patients and at low frequencies across healthy donors. Accordingly, in certain embodiments, the compositions and methods disclosed herein are used to guide clinical decision making, e.g., identification of prognostic factors, monitoring of treatment response or disease progression, or implementation of preventative measures.

Information about the immunoprevalent T cell epitopes associated with T1D can also be used to determine whether a subject may elicit a more desirable immune response to one therapeutic agent over another. In certain embodiments, a subject having CD8+ T cells (e.g., cytotoxic T cells) reactive to a given T1D-associated epitope is treated with a therapy that comprises the epitope and/or a nucleic acid encoding the epitope.

Samples for making the assessment can be harvested for example from blood, body fluid, saliva, stool, or a tissue biopsy of a patient. The samples can be analyzed the presence and/or amount of T cells reactive with the T cell epitopes using a peptide-MHC tetramer assay, an Enzyme Linked Immuno Spot Assay (ELISpot), or an Activation Induced Marker (AIM) assay. The methods can be conducted in a multiplex manner. For example, a sample of T cells from a patient can be contacted with a peptide-MHC multimer library comprising a plurality of the T cell epitopes disclosed herein, and T cells in the sample that binds to at least one member of the peptide-MHC multimer library can be identified. The method can further comprise determining the sequence of the peptide(s) loaded onto the MHC multimer(s) to which the T cell binds, to thereby determine the antigenic specificity of the T cell response in the patient. It is contemplated that such a method can be conducted on a plurality of patients, and the resulting information can be used to identify a patient subpopulation having an antigen-specific T cell response of interest.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein.

It should be understood that the order of steps or order for performing certain actions is immaterial so long as the present disclosure remain operable. Moreover, two or more steps or actions may be conducted simultaneously.

The description above describes multiple aspects and embodiments of the disclosure. The patent application specifically contemplates all combinations and permutations of the aspects and embodiments.

### EXAMPLES

The disclosure now being generally described, will be more readily understood by reference to the following examples, which are included merely for purposes of illustration of certain aspects and embodiments of the present disclosure, and is not intended to limit the disclosure.

### Example 1. Identification of Immunogenic T Cell Epitopes Associated with T1D

This example provides an overview of how immunogenic T cell epitopes associated with T1D were identified by a series of screens and validation assays.

It is known in the field that there are T cells circulating in the periphery of both T1D patients and healthy donors that recognize T cell epitopes possibly associated with T1D. Due to the small number of known epitopes, the relative immunoprevalence of cognate T cells and disease relevance of any specific epitope is difficult to establish. First, a large number of potential T1D-associated epitopes that may be presented by a specific MHC allele was profiled. Second, a comparative analysis of the cognate T cell repertoire in T1D patients and healthy donor controls on an epitope-by-epitope basis was performed.

Briefly, a target gene set was defined. Thousands of antigens were known to be expressed and enriched in beta cells, which incorporate potentially millions of epitopes. To refine this pool of potential antigens, antigens with known epitopes were included, such as non-germline encoded epitopes including but not limited to post translationally modified peptides, defective ribosomal products, mimotopes, and hybrid insulin peptide epitopes (James et al., (2020) Diabetes 69:1311-1335, IEDB database www.iedb.org). Also included were beta cell overexpressed genes identified in (Gonzalez-Duque et al., (2018) Cell Metab 28:946-960), interferon gamma, a known mediator of cytotoxic and inflammatory immune responses, other upregulated genes identified in (Eizirik et al., (2012) PLOS Genet 8: e1002552), and a custom bioinformatics analysis of publicly available datasets to further identify beta cell antigens that may or may not be shared in autoimmune diseases genetically linked to T1D, such as Celiac disease. Additional beta cell antigens were identified from RNA sequencing datasets from the Genotype Tissue Expression (GTEx) project and from a study of pancreatic cell-type-specific gene expression (www.gtexportal.org/home/datasets, Nica et al., (2013) Genome Res 23:1554-1562). From this combined antigen pool, several dozens of antigens and several hundred to a thousand specific epitopes were selected based on in silico prediction (Reynisson et al., (2020) Nucleic Acids Res 48: W449-W454) and a custom bioinformatic workflow. A library of barcoded peptide-MHC proteins was then created and used for high-throughput screening of T cell-pMHC interactions in tens of millions of primary T cells isolated from peripheral blood mononuclear cells (PBMCs) samples drawn from cohorts of patients with T1D and healthy donor controls. The epitopes in the library were filtered for relevance to T1D on the basis of their average prevalence in the T1D cohort, the extent that they were observed across all T1D patients, and the relative enrichment in abundance in T1D patients compared to healthy controls.

The large dataset enabled the identification of highly immunoprevalent epitopes that are over-represented in the periphery of patients with T1D. It reduced a starting set of millions of possible beta cell associated T cell epitopes to several tens or hundreds of highly relevant epitopes.

### Example 2. Binding of T1D-Associated Epitopes to Patient Peripheral T Cells

This example describes assessment of peptide binding to peripheral T cells from 8 short clinical duration T1D patients and 21 healthy donors who are HLA-matched without a history of diabetes, thereby to identify T1D-associated, HLA-A*02:01-restricted T cell epitopes.

Briefly, in the high-throughput screen described in Example 1, a library of HLA-A*02:01 oligo-barcoded pMHC tetramers was created as described in Francis et al., (2022) Sci Immunol 21:eabk3070. This tetramer library was composed of individual pMHCs representing hundreds of different epitopes identified in Example 1. CD8+ T cells were derived from frozen peripheral blood mononuclear cells (PBMCs) of human donors. CD8+ T cells were enriched via magnetic-activated cell separation from thawed PBMCs via commercially available negative enrichment kits (Miltenyi). Resulting enriched CD8+ T cells were counted and a standard input of 1 million CD8+ T cells per subject were then stained with tetramer libraries at final concentration of 1 nM per pMHC tetramer, in the presence of 2 mg/mL salmon sperm DNA in PBS with 0.5% BSA solution for 20 min. Stained cells were barcoded by commercially available anti-TCR antibody-derived tags (ADT, clone IP26, Biolegend). Tetramer bound cells were then labeled with phycoerythrin (PE) conjugated anti-DKDDDDK-Flag antibody (Biolegend) followed by dead cell discrimination using 7-amino-actinomycin D (7-AAD). Live, tetramer positive cells were sorted using a Sony MA900 Sorter. Following sorting, tetramer positive cells were single-cell sequenced using commercially available systems and protocols from 10× Genomics. Post-sequencing cells were demultiplexed and passed through standard quality control workflows (removal of duplicates, damaged cells, empty droplets, etc.) as described in Francis et al., (2022) Sci Immunol 21:eabk3070. Demultiplexed, tetramer positive cells were scored for pMHC-TCR interactions as described in Francis et al., (2022) Sci Immunol 21:eabk3070. Non-control pMHC-TCR putative binders passing quality control filters and scoring were used for analysis. The resulting pMHC-TCR matrix reactivity map was analyzed by abundance for “normalized T1D prevalence”, “relative enrichment”, and “% presence in T1D samples.” Normalized prevalence was defined as the average number of times an epitope in the pMHC tetramer library was bound by cognate TCR in the T1D cohort. Relative enrichment was defined as an epitope significantly increased in T1D compared to healthy, as determined by having a p value of less than 0.05 in a two-tailed t-Test that assumes an unequal variance. Finally, for an epitope to be considered of value, it must pass prevalence and enrichment thresholds and have cognate T cells present in at least 75 percent of T1D subjects. Table 4 shows normalized T1D prevalence, relative enrichment in T1D patients as a p value, and the percentage of T1D samples in which the epitope response was observed, of selected epitopes.

TABLE 4
Binding of T1D-associated epitopes to PBMC-derived T cells from T1D patients

| | | Cognate T cells | | |
|---|---|---|---|---|
| | | | Relative | |
| | T cell epitope | Normalized T1D | enrichment | % Presence in |
| Antigen | sequence | prevalence | T1D/HD p-value | T1D samples |
| PTPRN2 | MVWESGCVV | 16.313 | 0.002 | 100 |
| G6PC2 | VIGDWLNLI | 2.188 | 0.025 | 75 |
| CHGA | TLSKPSPMPV | 1.06 | 0.004 | 87.5 |
| CYP27B1 | TLVTLCHYA | 1.94 | 0.005 | 87.5 |
| PCSK2 | KMAKDWKTV | 2.38 | 0.006 | 100 |
| RASGRP1 | TLMAVIGGL | 3.63 | 0.006 | 100 |
| CCNI | KLSPSQHLA | 1.06 | 0.008 | 87.5 |
| EGR4 | YAPWELLSV | 2.44 | 0.008 | 87.5 |
| CCNI | LQFRGSMLA | 2.00 | 0.010 | 100 |
| C1ORF127 | ALAWAVWLA | 1.94 | 0.010 | 87.5 |
| GCG | FVAGLFVML | 1.88 | 0.012 | 87.5 |
| KIF1A | VLQASSISA | 3.19 | 0.015 | 100 |
| IFIH1 | VLVNKVLLV | 1.69 | 0.016 | 87.5 |
| INS | SVALWNNAV | 1.13 | 0.017 | 87.5 |
| PTPRN2 | FSESILTYV | 2.88 | 0.021 | 87.5 |
| PTPRN | FLPYDHARI | 2.94 | 0.021 | 87.5 |
| INS | ALMGQAAGV | 1.50 | 0.023 | 87.5 |
| EGR4 | LMSGILGLA | 2.44 | 0.023 | 87.5 |
| EGR4 | FLSWALNSC | 2.50 | 0.026 | 100 |
| KIF1A | NLLYPVPLV | 2.81 | 0.027 | 87.5 |
| CYP27B1 | TLSWALYEL | 2.50 | 0.028 | 100 |
| PDX1 | GQWAGGAYA | 1.81 | 0.035 | 87.5 |
| CCNI | KLNWDLHTA | 3.63 | 0.042 | 87.5 |
| BMP5 | QALDVGWLV | 1.19 | 0.043 | 87.5 |
| SH2B3 | NLYTFVLKV | 2.56 | 0.049 | 87.5 |

As shown in Table 4, the epitopes listed in Table 4 represent HLA-A*02:01 tetramer library members with sufficient pMHC-TCR interactions in the peripheral blood of individuals with T1D to pass filtering thresholds for normalized T1D prevalence, relative enrichment, and are shared in greater than or equal to 75% of T1D subjects. The other epitopes in Tables 1-3 also exhibited at least 0.5 normalized T1D prevalence and were shared in at least 50% the T1D patients assessed.

### Example 3. Binding of T1D-Associated Epitopes to Patient Pancreatic Islet T Cells

This example describes assessment of peptides that bind to T cell lines derived from living pancreatic islets of recently deceased pancreatic organ donors with T1D, thereby to identify T1D-associated, HLA-A*02:01-restricted T cell epitopes directly associated with islet infiltrating T cells. This assay, using specialized post-mortem processing of difficult-to-obtain pancreas biopsy, further confirmed the association of selected epitopes with T1D, given that disease-associated T cell epitopes generally result in infiltration of T cells that can cause inflammation and subsequent beta cell cytotoxicity in the pancreas.

Briefly, pancreatic slices were sourced from individuals with suspected or confirmed Type I diabetes diagnosis at the time of death by the Network of Pancreatic Organ Donors. Within in 72 hours of death, pancreatic islets were macrodissected from the slices under sterile conditions. These islets were cultured with a cocktail of cytokines and immune stimulating factors that allow T cell expansion independent of cognate TCR-pMHC interaction, using the polyclonal T cell expansion method described in Babon et al., (2016) Nat Med 22:1482-1487. The resulting T cell lines were then screened against pMHC libraries described in Example 2.

TABLE 5
Binding of T1D-associated epitopes to islet-
derived T cells from T1D patients

| Antigen | T cell epitope |
|---|---|
| G6PC2 | VIGDWLNLI |

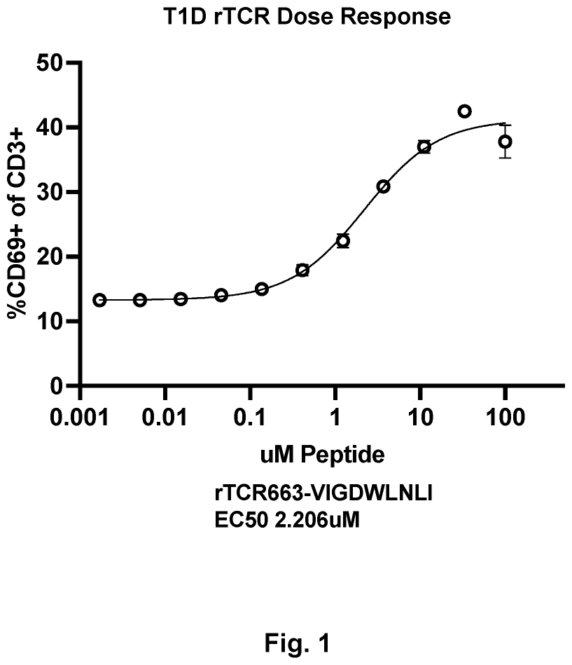

Table 5 shows a G6PC2 epitope that was identified as binding the polyclonally expanded pancreatic islet infiltrating T cells. This epitope bound to a single, high abundance TCR derived from an individual that was diagnosed with Type I diabetes 4 months prior to death. As shown in FIG. 1, an islet-derived rTCR was expressed in Jurkat T cells that were pulsed with 0.001 to 100 μM of the G6PC2 epitope peptide, and an EC50 (half maximal effective concentration) of 2.2 μM could be determined from the dose-response curve. The EC50 value represents an approximate affinity for the pMHC-rTCR-VIG-islets interaction.

### Example 4. Stimulation of TCR Recombinant T Cells by T1D-Associated Epitopes

This example describes functional validation of the T cell epitopes described in Examples 2 and 3, and their cognate TCRs, by assessing the ability of certain T cell epitopes, when presented by APCs that express HLA-A*02:01, to activate an immortalized T cell line expressing recombinant TCRs sequenced from the T cells of T1D patients and healthy donors. The analysis of peripheral T cells and beta islet infiltrating T cell lines resulted in the identification of T1D associated T cell epitopes of high relevance to T1D disease. The relevance of certain identified T1D associated epitopes was evidenced by binding and activation of cognate TCRs in primary T cells or recombinant T cell lines.

Briefly, native paired alpha and beta TCR sequences identified from human tissues, such as PBMC or islet-derived CD8 T cells, were cloned into the pLVX-EFla lentiviral backbone (Takara) as a bicistronic TCRB-T2A-TCRa vector. TCRa/B/Jurkat J76 cells, which were engineered to express CD8 and a nuclear factor of activated T cells (NFAT)-green fluorescent protein (GFP) reporter (referred to as J76-CD8-NFAT-GFP), were transduced with lentiviral vectors produced from HEK 293T cells. To assess functional activity of the recombinant TCRs (rTCRs), J76-CD8-NFAT-GFP expressing recombinant TCRs were incubated at a 1:1 ratio with the HLA-A*02:01⁺ T2(174×CEM.T2) (ATCC CRL-1991) lymphoblastic cell line, with a final concentration of 50 μM of cognate peptide (Vivitide, >95% pure) or 0.5% dimethylsulfoxide (DMSO, vehicle). Cell mixtures were incubated at 37° C., 5% CO₂ for 16 hours. Activation of the T cells was assessed by flow cytometry using anti-CD3-PE-Cy7 (Clone UCHT1) and anti-CD69-APC (Clone FN50) antibodies, and analyzed using the Satorius iQue Screener Plus and FlowJo v10. Activation of rTCR-expressing cell lines was determined by comparing the mean percentage of CD69⁺ cells out of CD3⁺ cells, in cell mixtures with cognate peptide-loaded T2 cells, with the mean percentage in cell mixtures with no peptide (DMSO control).

TABLE 6
T cell activation of by T1D-associated epitopes

| | | T Cell Activation | | | |
|---|---|---|---|---|---|
| | | | | Mean % | Mean % |
| | | | | CD69+ T | CD69+ T |
| | | | | cells with | cells with |
| | T Cell Epitope | | Source | cognate | DMSO |
| Antigen | Sequence | TCR | of TCR | peptide | control |
| CYP27B1 | TLSWALYEL | TCR_01 | PBMC | 59.8 | 9.045 |
| CYP27B1 | TLSWALYEL | TCR_02 | PBMC | 58 | 5.725 |
| KIF1A | NLLYPVPLV | TCR_03 | PBMC | 49.75 | 7.495 |
| CYP27B1 | TLSWALYEL | TCR_04 | PBMC | 48 | 9.92 |
| EGR4 | YAPWELLSV | TCR_05 | PBMC | 38.4 | 3.48 |
| CYP27B1 | TLSWALYEL | TCR_06 | PBMC | 39 | 5.275 |
| CYP27B1 | TLSWALYEL | TCR_07 | PBMC | 33.55 | 4.805 |
| KIF1A | NLLYPVPLV | TCR_08 | PBMC | 27.25 | 7.16 |
| G6PC2 | VIGDWLNLI | TCR_09 | PBMC | 26.15 | 4.745 |
| IFIH1 | VLVNKVLLV | TCR_10 | PBMC | 29.45 | 6.57 |
| KIF1A | NLLYPVPLV | TCR_11 | PBMC | 21.65 | 7.3 |
| PCSK2 | KMAKDWKTV | TCR_12 | PBMC | 18.65 | 5.38 |
| EGR4 | YAPWELLSV | TCR_13 | PBMC | 17.55 | 8.075 |
| CYP27B1 | TLSWALYEL | TCR_14 | PBMC | 15.45 | 4.995 |
| PTPRN2 | LLPPRVLPA | TCR_15 | PBMC | 32.25 | 10.4 |
| CCNI | QLLFSLPKL | TCR_16 | PBMC | 61.55 | 6.38 |
| CPA1 | TLDIFLEIV | TCR_17 | PBMC | 36.7 | 5.63 |
| G6PC2 | VIGDWLNLI | TCR_18 | PBMC | 26.15 | 4.745 |
| G6PC2 | VIGDWLNLI | TCR_19 | islet | 28.8 | 4.05 |

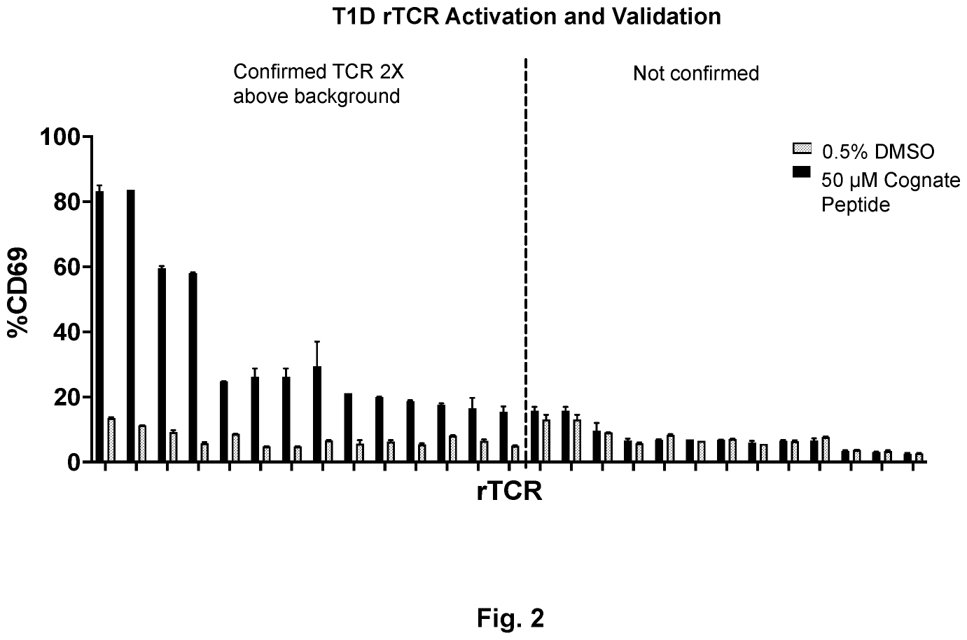

Table 6 and FIG. 2 (left side of dashed line) shows HLA-A*02:01 restricted epitopes that were identified to have cognate TCR reactivity in T1D PBMCs and/or islet-derived primary T cells. The corresponding rTCR-expressing cell lines had 2× or higher activation relative to control samples without peptide loading.

### Example 5. Validation of T1D-Associated Epitopes in Adult Onset T1D Cohorts

This example assesses the prevalence of epitopes listed in Tables 1-3 and to confirm the validity of these epitopes as therapeutic targets of therapeutic and/or prophylactic T1D modalities. In this experiment, epitopes listed in Tables 1-3 will be evaluated in human PBMCs isolated from a cohort of adult-onset T1D, in which the T1D patients will be further classified by time from T1D onset, e.g., new-onset (PBMCs isolated <100 days from disease onset) and longstanding or advanced disease (PBMCs isolated >5 years from disease onset). The results will be compared to healthy adults that are matched for age and sex, and further compared to pediatric-onset T1D matched for age and sex.

T cell reactivity and activation of the T cells isolated from these patients will be assessed as described in Example 4. The results of this experiment are anticipated to confirm the increased recognition of these epitopes by autoreactive CD8+ T cells (e.g., Table 4). Further, these results will be used to refine the targeting of therapeutic agents for the treatment of new-onset T1D and prophylactic applications in autoimmune diabetes that have yet to progress to clinical T1D, e.g. stage II autoantibody positive autoimmune diabetes. Moreover, the data generated from this experiment will be used for confirmation experiments to determine the specificity of TCRs identified in the experiment (e.g. Table 6). These results will provide further evidence of the efficacy and utility of the pharmaceutical compositions comprising T1D-associated peptides disclosed herein.

### Example 6. Comparison of the Reactivity of PBMC-Derived T Cell and Pancreatic T Cell to T1D-Associated Epitopes

This example confirms the presence of T cells in the peripheral blood and pancreatic tissue of human T1D patients that are reactive to the epitopes described in Tables 1-3. The TCR reactivities and sequence(s) of CD8+ T cells isolated from PBMCs are compared to T cells isolated from pancreatic tissue, specifically insulin producing beta-islet T cells and T cells derived from the draining pancreatic lymph nodes. In this experiment, T cells are isolated from the PBMCs of T1D patients as described in Example 2, and from the islets of deceased T1D patients as described in Example 3. T cell reactivity and activation of the T cells isolated from these patients will be assessed as described in Example 4. This study will further support the existence and clinical utility of autoreactive CD8+ T cells specific for the epitopes listed in Tables 1-3.

### INCORPORATION BY REFERENCE

All publications and patents cited throughout the text of this specification (including all patents, patent applications, scientific publications, manufacturer's specifications, instructions, etc.) are hereby incorporated by reference in their entirety for all purposes. To the extent the material incorporated by reference contradicts or is inconsistent with this specification, the specification will supersede any such material.

### EQUIVALENTS

The disclosure may be embodied in other specific forms without departing from the spirit or essential characteristics thereof. The foregoing embodiments are therefore to be considered in all respects illustrative rather than limiting the disclosure described herein. The scope of the disclosure is thus indicated by the appended claims rather than by the foregoing description, and all changes that come within the meaning and range of equivalency of the claims are intended to be embraced therein.

What is claimed is:

1. A pharmaceutical composition comprising:
(a) an isolated T1D-associated peptide comprising an amino acid sequence set forth in Table 2, Table 1, or Table 3, or a nucleic acid encoding the T1D-associated peptide; and
(b) a vehicle for targeting the T1D-associated peptide or the nucleic acid to a target cell.

2. The pharmaceutical composition of claim 1, wherein the T1D-associated peptide comprises 8-100, 8-50, 8-30, 8-25, 8-20, 8-15, 15-100, 15-50, 15-30, 15-25, or 15-20 contiguous amino acids of the corresponding antigen set forth in Table 2, Table 1, or Table 3.

3. The pharmaceutical composition of claim 1 or 2, wherein the vehicle comprises a delivery agent for delivering the T1D-associated peptide or the nucleic acid into the target cell.

4. The pharmaceutical composition of claim 3, wherein the T1D-associated peptide comprises at least 70%, at least 80%, at least 90%, or at least 95% of the entire length of the corresponding antigen.

5. The pharmaceutical composition of claim 3 or 4, wherein element (a) comprises the T1D-associated peptide.

6. The pharmaceutical composition of claim 5, wherein the delivery agent comprises a nanoparticle.

7. The pharmaceutical composition of claim 6, wherein the nanoparticle comprises a liposome.

8. The pharmaceutical composition of claim 3 or 4, wherein element (a) comprises the nucleic acid encoding the T1D-associated peptide.

9. The pharmaceutical composition of claim 8, wherein the nucleic acid comprises a messenger RNA (mRNA).

10. The pharmaceutical composition of claim 8 or 9, wherein the delivery agent comprises a lipid nanoparticle.

11. The pharmaceutical composition of any one of claims 3-10, wherein the target cell is a dendritic cell, a macrophage, a B cell, or a non-professional antigen presenting cell (APC).

12. The pharmaceutical composition of any one of claims 3-11, wherein the target cell is a tolerogenic cell.

13. The pharmaceutical composition of claim 12, wherein the tolerogenic cell is a cell in the liver, optionally selected from a liver sinusoidal endothelial cell, a MARCO⁺ Kupffer cell, and a monocyte-derived macrophage.

14. The pharmaceutical composition of claim 12, wherein the tolerogenic cell is a cell in the spleen, optionally selected from a marginal zone macrophage, a metallophilic macrophage, and a marginal zone dendritic cell.

15. The pharmaceutical composition of any one of claims 3-14, wherein the delivery agent has a negative zeta potential.

16. The pharmaceutical composition of any one of claims 3-15, wherein the delivery agent comprises a ligand that binds the target cell.

17. The pharmaceutical composition of any one of claims 3-16, further comprising an immunomodulator.

18. The pharmaceutical composition of claim 17, wherein the immunomodulator comprises an immunomodulatory cytokine or a nucleic acid encoding the immunomodulatory cytokine.

19. The pharmaceutical composition of claim 18, wherein the immunomodulatory cytokine is selected from IL-2, IL-10, TGF-β, IL-37, IL-27, IL-35, IL-31, and Vasoactive Intestinal Peptide (VIP), and variants thereof.

20. The pharmaceutical composition of claim 19, wherein the immunomodulatory cytokine is a mutant IL-2 that preferentially activates the IL-2Rαβγ receptor complex relative to the IL-2Rβγ receptor complex.

21. The pharmaceutical composition of any one of claims 17-20, wherein the immunomodulator comprises or further comprises a nucleic acid encoding an intracellular or transmembrane immunomodulatory protein.

22. The pharmaceutical composition of claim 21, wherein the intracellular or transmembrane immunomodulatory protein is selected from the group consisting of PD-L1, PD-L2, ICOS ligand, ILT3, ILT4, BTLA, Fas, CD39, and indoleamine 2,3-dioxygenase 1 (IDO1).

23. The pharmaceutical composition of any one of claims 17-22, wherein the immunomodulator comprises or further comprises an immunomodulatory compound.

24. The pharmaceutical composition of claim 23, wherein the immunomodulatory compound is selected from the group consisting of vitamin A, vitamin D, adenosine, kynurenine, and 2-(1′ H-indole-3′-carbonyl)-thiazole-4-carboxylic acid methyl ester (ITE).

25. The pharmaceutical composition of any one of claims 17-24, wherein the immunomodulator is in or on the delivery agent.

26. The pharmaceutical composition of claim 1 or 2, wherein element (a) comprises a peptide-MHC complex comprising the T1D-associated peptide and a cognate class I MHC, and the vehicle comprises a presentation agent that binds a plurality of the peptide-MHC complexes.

27. The pharmaceutical composition of claim 26, wherein the T1D-associated peptide consists of an amino acid sequence set forth in Table 1, Table 2, or Table 3.

28. The pharmaceutical composition of claim 26 or 27, wherein the class I MHC is HLA-A*02:01.

29. The pharmaceutical composition of any one of claims 26-28, wherein the class I MHC is a soluble class I MHC.

30. The pharmaceutical composition of claim 29, wherein the soluble class I MHC comprises an α1 domain and an α2 domain.

31. The pharmaceutical composition of claim 30, wherein the T1D-associated peptide binds the α1 domain and the α2 domain.

32. The pharmaceutical composition of claim 30 or 31, wherein the soluble class I MHC further comprises an α3 domain and a β2-microglobulin (β2m) subunit.

33. The pharmaceutical composition of claim 32, wherein the T1D-associated peptide is fused to the β2m subunit.

34. The pharmaceutical composition of any one of claims 26-33, wherein the peptide-MHC complex is on an outer surface of the presentation agent.

35. The pharmaceutical composition of claim 34, wherein the composition comprises an APC.

36. The pharmaceutical composition of claim 34, wherein the composition comprises an artificial antigen presenting cell (aAPC).

37. The pharmaceutical composition of claim 36, wherein the aAPC comprises a nanoparticle.

38. The pharmaceutical composition of any one of claims 26-33, wherein the presentation agent comprises a multimerization domain linked to the peptide-MHC complex.

39. The pharmaceutical composition of any one of claims 26-38, further comprising an immunomodulator.

40. The pharmaceutical composition of claim 39, wherein the immunomodulator comprises an immunomodulatory cytokine.

41. The pharmaceutical composition of claim 40, wherein the immunomodulatory cytokine is selected from IL-2, IL-10, TGF-β, IL-37, IL-27, IL-35, IL-31, and Vasoactive Intestinal Peptide (VIP), and variants thereof.

42. The pharmaceutical composition of claim 39, wherein the immunomodulator comprises an agonist of an immunomodulatory receptor of a T cell.

43. The pharmaceutical composition of claim 42, wherein the immunomodulatory receptor of the T cell is selected from PD-1, 4-1BB, CTLA-4, BTLA, LAG-3, TIM-3, TIGIT, CD2, or CD3.

44. The pharmaceutical composition of any one of claims 39-43, wherein the immunomodulator is conjugated to the peptide-MHC complex.

45. The pharmaceutical composition of any one of claims 39-43, wherein the immunomodulator is conjugated to the presentation agent.

46. The pharmaceutical composition of any one of claims 1-45, further comprising a pharmaceutically acceptable carrier or excipient.

47. A method of treating T1D, the method comprising administering to a subject in need thereof a therapeutically effective amount of the pharmaceutical composition of any one of claims 1-46.

48. The method of claim 47, wherein the subject expresses HLA-A*02:01.

49. The method of claim 47 or 48, wherein the method induces tolerance of the subject to the T1D-associated peptide.

50. A pharmaceutical composition comprising a Type 1 diabetes (T1D)-associated peptide comprising an amino acid sequence set forth in Table 2 or a nucleic acid encoding the T1D-associated peptide, wherein the peptide comprises 8-100, 8-50, 8-30, 8-25, 8-20, 8-15, 15-100, 15-50, 15-30, 15-25, or 15-20 contiguous amino acids of the corresponding antigen set forth in Table 2.

51. A pharmaceutical composition comprising a T1D-associated peptide comprising an amino acid sequence set forth in Table 1 or a nucleic acid encoding the T1D-associated peptide.

52. The pharmaceutical composition of claim 51, wherein the peptide comprises 8-100, 8-50, 8-30, 8-25, 8-20, 8-15, 15-100, 15-50, 15-30, 15-25, or 15-20 contiguous amino acids of the corresponding antigen set forth in Table 1.

53. Means for inducing a tolerogenic immune response in a subject in need thereof in combination with a pharmaceutically acceptable carrier.

54. A method of treating or preventing Type I diabetes in a subject in need thereof comprising administering to the subject an effective amount of a means for inducing a tolerogenic immune response in combination with a pharmaceutically acceptable carrier.


SEQUENCE LISTING (141 sequences)

SEQ ID NO: 1 - protein, 9 aa, synthetic construct
KLLEIMTRI

SEQ ID NO: 2 - protein, 9 aa, synthetic construct
FVAGLFVML

SEQ ID NO: 3 - protein, 9 aa, synthetic construct
LMSGILGLA

SEQ ID NO: 4 - protein, 9 aa, synthetic construct
QALDVGWLV

SEQ ID NO: 5 - protein, 9 aa, synthetic construct
YAPWELLSV

SEQ ID NO: 6 - protein, 9 aa, synthetic construct
ALAWAVWLA

SEQ ID NO: 7 - protein, 9 aa, synthetic construct
FLSWALNSC

SEQ ID NO: 8 - protein, 9 aa, synthetic construct
GQWAGGAYA

SEQ ID NO: 9 - protein, 9 aa, synthetic construct
NLYTFVLKV

SEQ ID NO: 10 - protein, 9 aa, synthetic construct
TLMAVIGGL

SEQ ID NO: 11 - protein, 9 aa, synthetic construct
TLSWALYEL

SEQ ID NO: 12 - protein, 9 aa, synthetic construct
VLVNKVLLV

SEQ ID NO: 13 - protein, 9 aa, synthetic construct
TLVTLCHYA

SEQ ID NO: 14 - protein, 9 aa, synthetic construct
KMNGRPAFI

SEQ ID NO: 15 - protein, 9 aa, synthetic construct
AQTNSFFML

SEQ ID NO: 16 - protein, 9 aa, synthetic construct
GLLVLSVLL

SEQ ID NO: 17 - protein, 9 aa, synthetic construct
HMNATNHAI

SEQ ID NO: 18 - protein, 9 aa, synthetic construct
KMIKCVIPG

SEQ ID NO: 19 - protein, 9 aa, synthetic construct
LLLVALSVA

SEQ ID NO: 20 - protein, 9 aa, synthetic construct
RLAAPLAVL

SEQ ID NO: 21 - protein, 9 aa, synthetic construct
SIIKAIYQA

SEQ ID NO: 22 - protein, 9 aa, synthetic construct
SLEAACPPV

SEQ ID NO: 23 - protein, 9 aa, synthetic construct
TILERLLEA

SEQ ID NO: 24 - protein, 9 aa, synthetic construct
TLWIPRSHV

SEQ ID NO: 25 - protein, 9 aa, synthetic construct
TMIGRILLT

SEQ ID NO: 26 - protein, 9 aa, synthetic construct
YMTLWIPRS

SEQ ID NO: 27 - protein, 9 aa, synthetic construct
TLDIFLEIV

SEQ ID NO: 28 - protein, 9 aa, synthetic construct
YVDMNATTV

SEQ ID NO: 29 - protein, 11 aa, synthetic construct
ALSTCYIMCMV

SEQ ID NO: 30 - protein, 9 aa, synthetic construct
ALRPYLPAL

SEQ ID NO: 31 - protein, 9 aa, synthetic construct
AMPHWLRHL

SEQ ID NO: 32 - protein, 9 aa, synthetic construct
ASFPFSFCV

SEQ ID NO: 33 - protein, 9 aa, synthetic construct
CMEEELLTI

SEQ ID NO: 34 - protein, 9 aa, synthetic construct
FLPEWTGIA

SEQ ID NO: 35 - protein, 9 aa, synthetic construct
HLLTLSLDL

SEQ ID NO: 36 - protein, 9 aa, synthetic construct
KTAQDTLYV

SEQ ID NO: 37 - protein, 9 aa, synthetic construct
MMYKAIHCV

SEQ ID NO: 38 - protein, 9 aa, synthetic construct
RLLDPGCLV

SEQ ID NO: 39 - protein, 9 aa, synthetic construct
RMMVSLGHL

SEQ ID NO: 40 - protein, 9 aa, synthetic construct
SLGMLSVLA

SEQ ID NO: 41 - protein, 9 aa, synthetic construct
STPSFLAEL

SEQ ID NO: 42 - protein, 9 aa, synthetic construct
YLTFLPAEV

SEQ ID NO: 43 - protein, 9 aa, synthetic construct
FSDYQNYLV

SEQ ID NO: 44 - protein, 9 aa, synthetic construct
IMTIGTFGI

SEQ ID NO: 45 - protein, 9 aa, synthetic construct
RLSSYVVVV

SEQ ID NO: 46 - protein, 9 aa, synthetic construct
VVVDWASGV

SEQ ID NO: 47 - protein, 9 aa, synthetic construct
VIGDWLNLI

SEQ ID NO: 48 - protein, 9 aa, synthetic construct
KLSPSQHLA

SEQ ID NO: 49 - protein, 9 aa, synthetic construct
KMAKDWKTV

SEQ ID NO: 50 - protein, 9 aa, synthetic construct
LQFRGSMLA

SEQ ID NO: 51 - protein, 9 aa, synthetic construct
VLQASSISA

SEQ ID NO: 52 - protein, 9 aa, synthetic construct
FSESILTYV

SEQ ID NO: 53 - protein, 9 aa, synthetic construct
MVWESGCVV

SEQ ID NO: 54 - protein, 9 aa, synthetic construct
FLPYDHARI

SEQ ID NO: 55 - protein, 9 aa, synthetic construct
ALMGQAAGV

SEQ ID NO: 56 - protein, 9 aa, synthetic construct
SVALWNNAV

SEQ ID NO: 57 - protein, 9 aa, synthetic construct
NLLYPVPLV

SEQ ID NO: 58 - protein, 9 aa, synthetic construct
FLLLLLLLL

SEQ ID NO: 59 - protein, 9 aa, synthetic construct
ILLGLAWLA

SEQ ID NO: 60 - protein, 9 aa, synthetic construct
NLLFNIAKA

SEQ ID NO: 61 - protein, 9 aa, synthetic construct
VILIFPPMV

SEQ ID NO: 62 - protein, 9 aa, synthetic construct
ALSSRAASV

SEQ ID NO: 63 - protein, 9 aa, synthetic construct
AMVAALSPA

SEQ ID NO: 64 - protein, 9 aa, synthetic construct
CVFLWPFLL

SEQ ID NO: 65 - protein, 9 aa, synthetic construct
EMYDRAAEV

SEQ ID NO: 66 - protein, 9 aa, synthetic construct
FLDSDIPSV

SEQ ID NO: 67 - protein, 9 aa, synthetic construct
FLHIFHAIA

SEQ ID NO: 68 - protein, 9 aa, synthetic construct
FMTDIIEAS

SEQ ID NO: 69 - protein, 9 aa, synthetic construct
FQSESCPVV

SEQ ID NO: 70 - protein, 9 aa, synthetic construct
GIFETKFQV

SEQ ID NO: 71 - protein, 9 aa, synthetic construct
KVNFHMFDV

SEQ ID NO: 72 - protein, 9 aa, synthetic construct
LIDCAQYFL

SEQ ID NO: 73 - protein, 9 aa, synthetic construct
LMLHGTQSA

SEQ ID NO: 74 - protein, 9 aa, synthetic construct
QMSGSTTTI

SEQ ID NO: 75 - protein, 9 aa, synthetic construct
SLHERILFA

SEQ ID NO: 76 - protein, 9 aa, synthetic construct
SLLDRFLAT

SEQ ID NO: 77 - protein, 9 aa, synthetic construct
TLGKVISAL

SEQ ID NO: 78 - protein, 9 aa, synthetic construct
TLQAMADGV

SEQ ID NO: 79 - protein, 9 aa, synthetic construct
WLASGWTPA

SEQ ID NO: 80 - protein, 9 aa, synthetic construct
YLEHVQAVI

SEQ ID NO: 81 - protein, 9 aa, synthetic construct
YLHFLEPHT

SEQ ID NO: 82 - protein, 9 aa, synthetic construct
YQFNLYPET

SEQ ID NO: 83 - protein, 9 aa, synthetic construct
GLDLNVAEA

SEQ ID NO: 84 - protein, 9 aa, synthetic construct
YIKDGITRV

SEQ ID NO: 85 - protein, 9 aa, synthetic construct
FMAFVAMVT

SEQ ID NO: 86 - protein, 9 aa, synthetic construct
FSILLMEGV

SEQ ID NO: 87 - protein, 9 aa, synthetic construct
SLAVVTDAA

SEQ ID NO: 88 - protein, 9 aa, synthetic construct
LLPPRVLPA

SEQ ID NO: 89 - protein, 9 aa, synthetic construct
RLLQVPSSA

SEQ ID NO: 90 - protein, 9 aa, synthetic construct
TVADFWQMV

SEQ ID NO: 91 - protein, 9 aa, synthetic construct
AMDFYRYEV

SEQ ID NO: 92 - protein, 9 aa, synthetic construct
FLSWYDRGV

SEQ ID NO: 93 - protein, 9 aa, synthetic construct
LMAGLMQGV

SEQ ID NO: 94 - protein, 9 aa, synthetic construct
WQDDYTQYV

SEQ ID NO: 95 - protein, 9 aa, synthetic construct
ASLYHVYEV

SEQ ID NO: 96 - protein, 9 aa, synthetic construct
GVAGLLVAL

SEQ ID NO: 97 - protein, 9 aa, synthetic construct
ILAYMEDHL

SEQ ID NO: 98 - protein, 9 aa, synthetic construct
LLVALAVAL

SEQ ID NO: 99 - protein, 9 aa, synthetic construct
LTLVALAGV

SEQ ID NO: 100 - protein, 9 aa, synthetic construct
QLSTLLTLL

SEQ ID NO: 101 - protein, 9 aa, synthetic construct
TVIVMLTPL

SEQ ID NO: 102 - protein, 9 aa, synthetic construct
VLLEKKSPL

SEQ ID NO: 103 - protein, 9 aa, synthetic construct
VLLTLVALA

SEQ ID NO: 104 - protein, 9 aa, synthetic construct
LLMEGVPKS

SEQ ID NO: 105 - protein, 9 aa, synthetic construct
RTLTQFHFL

SEQ ID NO: 106 - protein, 9 aa, synthetic construct
SLADVTQQA

SEQ ID NO: 107 - protein, 9 aa, synthetic construct
WLIQISVCI

SEQ ID NO: 108 - protein, 9 aa, synthetic construct
YVLSFCKSA

SEQ ID NO: 109 - protein, 9 aa, synthetic construct
ALGIGTDSV

SEQ ID NO: 110 - protein, 9 aa, synthetic construct
FLIEEIERL

SEQ ID NO: 111 - protein, 9 aa, synthetic construct
KQKGFVPFL

SEQ ID NO: 112 - protein, 9 aa, synthetic construct
AMMIARFKM

SEQ ID NO: 113 - protein, 9 aa, synthetic construct
ALFHNATLL

SEQ ID NO: 114 - protein, 9 aa, synthetic construct
HMLHEPSHV

SEQ ID NO: 115 - protein, 9 aa, synthetic construct
RAEGCWAPA

SEQ ID NO: 116 - protein, 9 aa, synthetic construct
VVLQPAGPL

SEQ ID NO: 117 - protein, 9 aa, synthetic construct
WLDPRPQLC

SEQ ID NO: 118 - protein, 9 aa, synthetic construct
ALQVLCLPA

SEQ ID NO: 119 - protein, 9 aa, synthetic construct
HLQVLCLPA

SEQ ID NO: 120 - protein, 9 aa, synthetic construct
MLGPRWLYS

SEQ ID NO: 121 - protein, 9 aa, synthetic construct
NLYPETFAL

SEQ ID NO: 122 - protein, 9 aa, synthetic construct
QLLFSLPKL

SEQ ID NO: 123 - protein, 9 aa, synthetic construct
YMCTHRLLL

SEQ ID NO: 124 - protein, 9 aa, synthetic construct
MLMPVHFLL

SEQ ID NO: 125 - protein, 9 aa, synthetic construct
KLNWDLHTA

SEQ ID NO: 126 - protein, 9 aa, synthetic construct
ALLGIPLNV

SEQ ID NO: 127 - protein, 9 aa, synthetic construct
KMDQLAKEL

SEQ ID NO: 128 - protein, 9 aa, synthetic construct
AMSNLVPPV

SEQ ID NO: 129 - protein, 9 aa, synthetic construct
SVANALWIV

SEQ ID NO: 130 - protein, 10 aa, synthetic construct
RQFGPDWIVA

SEQ ID NO: 131 - protein, 10 aa, synthetic construct
RQWGPDPAAV

SEQ ID NO: 132 - protein, 10 aa, synthetic construct
TLSKPSPMPV

SEQ ID NO: 133 - protein, 9 aa, synthetic construct
IVEQCCTSI

SEQ ID NO: 134 - protein, 9 aa, synthetic construct
ALWGPDPAA

SEQ ID NO: 135 - protein, 10 aa, synthetic construct
RMMEYGTTMV

SEQ ID NO: 136 - protein, 9 aa, synthetic construct
SISVLISAL

SEQ ID NO: 137 - protein, 9 aa, synthetic construct
TMHSLTIQM

SEQ ID NO: 138 - protein, 9 aa, synthetic construct
YLLLRVLNI

SEQ ID NO: 139 - protein, 9 aa, synthetic construct
HIAGSLAVV

SEQ ID NO: 140 - protein, 9 aa, synthetic construct
RFKMFPEVK

SEQ ID NO: 141 - protein, 9 aa, synthetic construct
IVEQCCTSI